SEQUENCE LISTING (13 sequences)

SEQ ID NO: 1 - DNA, 21 bp, Artificial Sequence
  Synthetic oligonucleotide
tggaagaacaaaggtctctgg

SEQ ID NO: 2 - DNA, 19 bp, Artificial Sequence
  Synthetic oligonucleotide
gctggccaagagattggag

SEQ ID NO: 3 - DNA, 20 bp, Artificial Sequence
  Synthetic oligonucleotide
aggatcaccgtggctgctct

SEQ ID NO: 4 - DNA, 20 bp, Artificial Sequence
  Synthetic oligonucleotide
gggtgtgaaccatgagaagt

SEQ ID NO: 5 - DNA, 19 bp, Artificial Sequence
  Synthetic oligonucleotide
gactgtggtcatgagtcct

SEQ ID NO: 6 - DNA, 24 bp, Artificial Sequence
  Synthetic oligonucleotide
cagcaatgcctcctgcaccaccaa

SEQ ID NO: 7 - DNA, 23 bp, Artificial Sequence
  Synthetic oligonucleotide
agtttaagtttgaaattcgagcg

SEQ ID NO: 8 - DNA, 22 bp, Artificial Sequence
  Synthetic oligonucleotide
aaactaaataaaacgaacgcga

SEQ ID NO: 9 - DNA, 25 bp, Artificial Sequence
  Synthetic oligonucleotide
ggagtttaagtttgaaatttgagtg

SEQ ID NO: 10 - DNA, 32 bp, Artificial Sequence
  Synthetic oligonucleotide
ctaaaaactaaaaactaaataaaacaaacaca

SEQ ID NO: 11 - DNA, 19 bp, Artificial Sequence
  Synthetic oligonucleotide
aaaactgtgaacctgtggt

SEQ ID NO: 12 - DNA, 21 bp, Artificial Sequence
  Synthetic oligonucleotide
ttcaagcaaaagtgtgagaga

SEQ ID NO: 13 - DNA, 24 bp, Artificial Sequence
  Synthetic oligonucleotide
cagaacagtcaccaccaccttatt

(19) United States
(12) Patent Application Publication
(10) Pub. No.: US 2011/0124004 A1
(43) Pub. Date: May 26, 2011

(54) UTILITY OF HIGH MOLECULAR WEIGHT MELANOMA ASSOCIATED ANTIGEN IN DIAGNOSIS AND TREATMENT OF CANCER

(76) Inventors: Dave S.B. Hoon, Los Angeles, CA (US); Soldano Ferrone, Pittsburgh, PA (US); Minoru Kitago, (US); Yasufumi Goto, Matsumot (JP)

(21) Appl. No.: 12/981,501

(22) Filed: Dec. 30, 2010

Related U.S. Application Data
(62) Division of application No. 11/693,678, filed on Mar. 29, 2007.
(60) Provisional application No. 60/787,716, filed on Mar. 29, 2006.

(51) Int. Cl. C12Q 1/68; G01N 33/574

(52) U.S. Cl. 435/6.14; 435/7.23

(57) ABSTRACT

HMW-MAA antibody cocktails and their uses in detecting cancer and isolating cancer cells are disclosed. Also disclosed are methods of detecting cancer based on the presence of an HMW-MAA genomic sequence in circulating DNA, as well as the increased expression of the HMW-MAA gene and the reduced methylation of the HMW-MAA gene promoter in tissues and circulating cells.

### RELATED APPLICATION

This application is a divisional application of U.S. application Ser. No. 11/693,678, filed Mar. 29, 2007, which claims priority to U.S. Provisional Application Ser. No. 60/787,716, filed Mar. 29, 2006, the contents of which are incorporated herein by reference in their entirety.

### FIELD OF THE INVENTION

The present invention relates in general to high molecular weight melanoma associated antigen (HMW-MAA). More specifically, the invention relates to the utility of HMW-MAA in diagnosis and treatment of cancer.

### BACKGROUND OF THE INVENTION

The human HMW-MAA, also known as the melanoma chondroitin sulfate proteoglycan (MCSP), is a membrane-bound chondroitin sulfate proteoglycan that is highly expressed in human melanoma lesions and in a majority of human melanoma cell lines (1). HMW-MAA is also expressed in basal cell carcinoma (2), in several different types of tumors of neural crest origin, including astrocytoma, glioma, neuroblastoma, and in sarcomas (3-6). In addition, HMW-MAA is expressed in lobular breast carcinoma lesions (7). It is currently not known whether these findings reflect the presence of vascular pericytes in the surgically removed sections (8) or the expression of HMW-MAA by breast carcinoma cells.

HMW-MAA belongs to a family of adhesion receptors that mediate both cell-cell and cell-extracellular matrix interactions. Several lines of evidence suggest that HMW-MAA plays important roles in intracellular signal cascades important for cellular adhesion, spreading, and invasion (3, 9-12). These include the activation of small Rho family GTPase Cdc42 and of the adaptor protein p130cas (13), as well as the association of HMW-MAA with membrane-type 3 matrix metalloproteinase on melanoma cells (12). Furthermore, elevated HMW-MAA expression in early tumors has been proposed to facilitate tumor progression by enhancing the activation of focal adhesion kinase (FAK) and extracellular signal-regulated protein kinases 1 and 2 (ERK1/2) (14). The clinical relevance of these findings is indicated by the higher frequency of HMW-MAA expression in metastatic than in primary lesions in acral lentiginous melanoma (ALM), and by the association of HMW-MAA expression in primary ALM lesions with poor prognosis (15, 16). Furthermore, the role of HMW-MAA in the biology of melanoma cells may account for the statistically significant association between induction of HMW-MAA-specific antibodies and survival prolongation in patients with advanced melanoma immunized with HMW-MAA mimics (17, 18) and for the inhibition of human HMW-MAA-bearing melanoma tumor growth in SCID mice administered with HMW-MAA-specific monoclonal antibody (mAb) (19).

### SUMMARY OF THE INVENTION

This invention relates to methods for diagnosis and treatment of cancer based on the expression of the HMW-MAA gene in cancer cells.

In one aspect, the invention features a cocktail of antibodies to the HMW-MAA protein. The cocktail comprises at least two antibodies, each recognizing a distinct epitope on the HMW-MAA protein.

A cocktail of the invention can be used to detect the HMW-MAA protein. The HMW-MAA protein is contacted with a cocktail of the invention to allow binding of the HMW-MAA protein to its antibodies in the cocktail to form the HMW-MAA protein-antibody complexes. The HMW-MAA protein-antibody complexes are then detected.

A cocktail of the invention can also be used to detect cancer. Accordingly, the invention features a method of determining whether a subject is suffering from cancer. One step of the method involves providing a tissue or body fluid sample from a subject. The tissue is of a type susceptible to cancer or the metastasis of the cancer. The body fluid contains cells. The cancer is of a type in which the HMW-MAA protein is expressed. Another step of the method involves determination of the amount of the HMW-MAA protein in the sample with a cocktail of the invention. If the amount of the HMW-MAA protein in the sample is higher than a control amount, the subject is likely to be suffering from the cancer.

In another aspect, the invention features a device comprising a solid support and a cocktail of the invention immobilized on the solid support. A device of the invention can be used to isolate cells expressing the HMW-MAA protein. The method comprises (1) providing a device of the invention and a sample containing cells that express the HMW-MAA protein, (2) contacting the device with the sample to allow binding of the HMW-MAA protein to its antibodies, and (3) isolating the cells that express the HMW-MAA protein from the sample. In one embodiment, the sample is a cancer tissue sample or a sample of a body fluid containing cancer cells. The method may further comprise analyzing a DNA, mRNA, or protein marker in the isolated cells.

In a related aspect, the invention features a kit comprising a solid support and at least two antibodies to be immobilized on the solid support, each antibody recognizing a distinct epitope on the HMW-MAA protein. The kit can be used to make a device of the invention by immobilizing the HMW-MAA antibodies onto the solid support.

The invention further provides another method of determining whether a subject is suffering from cancer. The method involves providing a PE (paraffin-embedded) tissue sample from a subject. The tissue is of a type susceptible to cancer or the metastasis of the cancer. The cancer is of a type in which the HMW-MAA gene is expressed. The expression level of the HMW-MAA gene or the methylation level of the HMW-MAA gene promoter in the sample is determined. If the expression level of the HMW-MAA gene in the sample is higher than a control expression level, or if the methylation level of the HMW-MAA gene promoter in the sample is lower than a control methylation level, the subject is likely to be suffering from the cancer.

Another method of determining whether a subject is suffering from cancer comprises (1) providing a body fluid sample from a subject, wherein the sample contains DNA that exists as acellular DNA in the body fluid; and (2) detecting an HMW-MAA genomic sequence in the DNA. If the HMW-MAA genomic sequence is present in the DNA, the subject is likely to be suffering from cancer.

Also within the invention is still another method of determining whether a subject is suffering from cancer. The method comprises a step of providing a tissue or body fluid sample from a subject. The tissue is of a type susceptible to cancer or the metastasis of the cancer. The body fluid contains cells. The cancer is of a type in which the HMW-MAA gene is expressed. The method further comprises a step of determining the amount of the HMW-MAA mRNA in the sample. If the amount of the HMW-MAA mRNA in the sample is higher than a control amount, the subject is likely to be suffering from the cancer. The method may further comprise a step of determining the amount of the HMW-MAA protein in the sample using an antibody to the HMW-MAA protein or a cocktail of the invention. If the amount of the HMW-MAA protein in the sample is higher than a control amount, the subject is likely to be suffering from the cancer.

In particular, the invention provides a method of determining whether a subject is suffering from non-lobular breast cancer or pancreatic cancer. The method comprises a step of providing a tissue sample or a body fluid sample from a subject. The tissue is of a type susceptible to cancer or the metastasis of the cancer. The sample contains cellular DNA. The body fluid contains cells. The cancer is non-lobular breast cancer, or pancreatic cancer. The method additionally comprises a step of determining the expression level of the HMW-MAA gene in the sample or the methylation level of the HMW-MAA gene promoter in the DNA. If the expression level of the HMW-MAA gene in the sample is higher than a control expression level, or if the methylation level of the HMW-MAA gene promoter in the DNA is lower than a control methylation level, the subject is likely to be suffering from the cancer. The non-lobular breast cancer may be ductal or invasive breast cancer. Furthermore, the invention provides a method of reducing the expression level of a gene in a cell or subject. The method comprises contacting a non-lobular breast cancer or pancreatic cancer cell or a subject suffering from non-lobular breast cancer or pancreatic cancer with an agent that reduces the expression level of the HMW-MAA gene in the cell or subject.

The antibodies to the HMW-MAA protein may be selected from the group consisting of mAbs 225.28, 763.74, VT80.12, VF4-TP108, VF1-TP41.2, VF20-VT5.1, and TP61.5. A cocktail of the invention may include mAbs 225.28, 763.74, VF4-TP108, VF1-TP41.2, and TP61.5. Alternatively, a cocktail of the invention may include mAbs 763.74, VT80.12, and VF20-VT5.1.

In some embodiments of the invention, the cancer is melanoma, breast cancer, brain cancer, lung cancer, gastrointestinal cancer, sarcoma, or pancreatic cancer.

Exemplary solid supports include bead, gel, resin, microtiter plate, glass, and membrane.

The expression level of the HMW-MAA gene may be determined by detecting the HMW-HAA mRNA using qRT (quantitative real-time reverse transcription polymerase chain reaction), by detecting the HMW-MAA protein using an antibody to the HMW-MAA protein or a cocktail of the invention, or a combination thereof.

The invention provides reagents and methods for diagnosis and management of cancer with high specificity and sensitivity. The above-mentioned and other features of this invention and the manner of obtaining and using them will become more apparent, and will be best understood, by reference to the following description, taken in conjunction with the accompanying drawings. These drawings depict only typical embodiments of the invention and do not therefore limit its scope.

### BRIEF DESCRIPTION OF THE FIGURES

FIG. 1. A-F, Comparative IHC between MART-1 and HMW-MAA IHC in SLN macrometastasis. A, Melanoma cells are positive for anti-MART-1 Ab (×100). B, Melanoma cells are positive for anti-MART-1 Ab (×400). The cells immunoreactive for MART-1 show red cytoplasmic staining in melanoma cells. C, Melanoma cells are positive for anti-HMW-MAA Ab (×100). D, Melanoma cells are positive for anti-HMW-MAA Ab (×400). The cells staining for HMW-MAA show purple membrane staining in melanoma cells. E, Melanoma cells are negative for normal mouse IgG (×100). F, Melanoma cells are negative for normal mouse IgG (×400). G-L, Comparative IHC between MART-1 and HMW-MAA IHC in SLN macrometastasis of a melanoma patient. G, Melanoma cells are negative for anti-MART-1 Ab (×100). H, Melanoma cells are negative for anti-MART-1 Ab (×400). I, Melanoma cells are positive for anti-HMW-MAA Ab (×100). J, Melanoma cells are positive for anti-HMW-MAA Ab (×400). K, Melanoma cells are negative for normal mouse IgG (×100). L, Melanoma cells are negative for mouse IgG (×400). M-Q, Comparative IHC between MART-1 and HMW-MAA IHC in SLN micrometastasis of a melanoma patient. M, Melanoma cells are positive for anti-MART-1 Ab (×100). N, Melanoma cells are positive for anti-MART-1 Ab (×400). O, Melanoma cells are positive for anti-HMW-MAA Ab (×100). P, Melanoma cells are positive for anti-HMW-MAA Ab (×400). Q, Melanoma cells are negative for normal mouse IgG (×100). R, Melanoma cells are negative for normal mouse IgG (×400). S-X, Comparative IHC between MART-1 and HMW-MAA staining in SLN micrometastasis of a melanoma patient. S, Melanoma cells are negative for anti-MART-1 Ab (×100). T, Melanoma cells are negative for anti-MART-1 Ab (×400). U, Melanoma cells are positive for anti-HMW-MAA Ab (×100). V, Melanoma cells are positive for anti-HMW-MAA Ab (×400). W, Melanoma cells are negative for normal mouse IgG (×100). X, Melanoma cells are negative for normal mouse IgG (×400).

FIG. 2. A, HMW-MAA mRNA expression in melanoma cell lines and normal PBLs. HMW-MAA mRNA expression was designated as relative mRNA copies (absolute mRNA copies of HMW-MAA/absolute mRNA copies of GAPDH). The dotted bars indicate mean copy numbers. B; HMW-MAA mRNA expression in LN macrometastases, SLN micrometastases, and normal LNs. HMW-MAA mRNA expression was designated as relative mRNA copies (absolute mRNA copies of HMW-MAA/absolute mRNA copies of GAPDH). The dotted bars indicate mean copy numbers. The line is a cutoff line for HMW-MAA positivity at 2.95×10⁻². The cutoff point was above the mean relative HMW-MAA copy number plus 1 SD of normal LNs tissues. C, MART-1 mRNA expression in LN macrometastases, SLN micrometastases, and normal LNs. MART-1 mRNA expression was designated as relative mRNA copies (absolute mRNA copies of MART-1/absolute mRNA copies of GAPDH). The dotted bars indicate mean copy numbers.

FIG. 3 shows HMW-MAA expression in melanoma cells using cocktail mouse monoclonal ABs.

FIG. 4 shows distribution of IHC intensity of normal LN (n=15).

FIG. 5 shows frequency of IHC positive cells in normal LN (n=15).

FIG. 6 shows distribution of IHC intensity of SLN macrometastasis+micrometastasis (n=84).

FIG. 7 shows frequency of SLN macrometastasis+micrometastasis(+) by IHC (n=84).

FIG. 8 shows IHC comparison of SLN macrometastasis.

FIG. 9 shows HMW-MAA mRNA expression of melanoma cell lines by gel electrophoresis.

FIG. 10 shows HMW-MAA mRNA level of cell lines by qRT (copy number).

FIG. 11 shows HMW-MAA mRNA expression in SLN by qRT.

FIG. 12 shows isolation of melanoma cells from tumor biopsy specimens (direct method).

FIG. 13 is a flow chart for isolating melanoma cells from tumor biopsy specimens.

FIG. 14 shows melanoma cells captured by HMW-MAA beads.

FIG. 15 shows gel analysis of captured primary melanoma cells.

FIG. 16 shows blood HMW-MAA bead capture assay.

FIG. 17 shows results for normal healthy donors screened by HMW-MAA meads in 5 mL blood.

FIG. 18 shows multimarker mRNA expression in blood from stage III/IV melanoma patients.

FIG. 19 shows B-raf V600E mutant detection by PCR PNA/LNA clamping.

FIG. 20 shows B-raf V600E mutant DNA from circulating melanoma cells of stage III/IV patients.

FIG. 21 shows HMW-MAA IHC staining of breast cancer in case 1 (A, B) and case 2 (C).

### DETAILED DESCRIPTION OF THE INVENTION

The invention is based at least in part upon the unexpected discovery that HMW-MAA has utility as a more sensitive and specific biomarker than current common cancer biomarkers in melanoma. Accordingly, the invention provides a cocktail of antibodies to the HMW-MAA protein, which can be used to detect the HMW-MAA protein in cancer cells. An “antibody cocktail,” as used herein, is defined as a mixture of two or more antibodies, each recognizing a distinct epitope on an antigen. An “epitope” is a specific domain on an antigen that stimulates the production of, and is recognized by, an antibody.

Antibodies to the HMW-MAA protein and methods for producing such antibodies are well known in the art. See, e.g., Campoli M R, Chang C C, Kageshita T, Wang X, McCarthy J B, Ferrone S. Human high molecular weight-melanoma-associated antigen (HMW-MAA): a melanoma cell surface chondroitin sulfate proteoglycan (MSCP) with biological and clinical significance. Crit Rev Immunol 2004, 24:267-96. In general, an HMW-MAA protein or a fragment thereof can be used as an immunogen to generate antibodies using standard techniques for polyclonal and monoclonal antibody preparation. Typically, an antigenic peptide comprises at least 8 amino acid residues. An immunogen is used to prepare antibodies by immunizing a suitable subject (e.g., rabbit, goat, mouse, or other mammal) with the immunogen. The preparation can further include an adjuvant, such as Freund's complete or incomplete adjuvant, or similar immunostimulatory agent. Immunization of a suitable subject with an immunogenic preparation induces a polyclonal antibody response. The antibody titer in the immunized subject can be monitored over time by standard techniques, such as an enzyme linked immunosorbent assay (ELISA) using immobilized HMW-MAA. If desired, the antibody molecules directed against HMW-MAA can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96), or trioma techniques. Alternative, a monoclonal antibody can be identified and isolated by screening a recombinant combinatorial immunoglobulin library (e.g., an antibody phage display library) with an antigen to isolate immunoglobulin library members that bind to the antigen. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example, using methods described in Better et al. (1988) Science 240:1041-1043; Liu et al. (1987) Proc. Natl. Acad. Sci. USA 84:3439-3443; Nishimura et al. (1987) Canc. Res. 47:999-1005.

The term “antibody” refers to immunoglobulin molecules and immunologically active portions thereof, i.e., molecules that contain an antigen binding site which specifically binds an antigen. A molecule which specifically binds to HMW-MAA is a molecule which binds HMW-MAA, but does not substantially bind other molecules in a sample, e.g., a biological sample. Examples of immunologically active portions of immunoglobulin molecules include F(ab) and F(ab′)₂ fragments which can be generated by treating the antibody with an enzyme such as pepsin.

In some embodiments of the invention, the antibodies to the HMW-MAA protein are selected from the group consisting of mAbs 225.28, 763.74, VT80.12, VF4-TP108, VF1-TP41.2, VF20-VT5.1, and TP61.5. For example, a cocktail of the invention may include a five-member combination of mAbs 225.28, 763.74, VF4-TP108, VF1-TP41.2, and TP61.5 or a three-member combination of mAbs 763.74, VT80.12, and VF20-VT5.1.

A cocktail of the invention may be immobilized onto a solid support to form a device which, in turn, can be used to isolating cells (e.g., cancer cells) expressing the HMW-MAA protein. The solid support may take any convenient form such as beads, gels, resins, microtiter plates, glass, and membranes. The support may be composed of any material on which antibodies are conventionally immobilized, e.g., nitrocellulose, polystyrene, and polyvinyl chloride.

An antibody may be immobilized onto the solid support by any conventional means, e.g., absorption, covalent binding with a cross-linking agent, and covalent linkage resulting from chemical activation of either the solid support or the antibody or both. The immobilization of the antibody may be accomplished by immobilizing one half of a binding pair, e.g., streptavidin, to the solid support and binding the other half of the same binding pair, e.g., biotin, to the antibody. Suitable means for immobilizing an antibody onto a solid support are disclosed in the Pierce Catalog, Pierce Chemical Company, P.O. Box 117, Rockford, III. 61105, 1994.

In some embodiments, the solid support is blocked to reduce or prevent the non-specific binding of a target cell to the solid support. Any conventional blocking agents can be used. Suitable blocking agents are described in U.S. Pat. Nos. 5,807,752; 5,202,267; 5,399,500; 5,102,788; 4,931,385; 5,017,559; 4,818,686; 4,622,293; and 4,468,469. Exemplary blocking agents include goat serum, bovine serum albumin, and milk proteins (“blotto”). The solid support may be blocked by absorption of the blocking agent either prior to or after immobilization of an antibody. Preferably, the solid support is blocked by absorption of the blocking agent after immobilization of the antibody. The exact conditions for blocking the solid support, including the exact amount of the blocking agent used, depend on the identities of the blocking agent and the solid support but may be easily determined using the assays and protocols well known in the art.

Antibodies to the HMW-MAA protein and a solid support may be included in a kit. The kit contains at least two antibodies, each recognizing a distinct epitope on the HMW-MAA protein. The antibodies can be immobilized onto the solid support using the methods described above to make a device of the invention.

A cocktail of the invention can be used to detect the HMW-MAA protein (e.g., in a cellular lysate or cell supernatant, or on an in situ cell) in order to evaluate the abundance and pattern of the expression of the HMW-MAA protein. This method generally involves contacting the HMW-MAA protein with a cocktail of the invention to allow binding of the HMW-MAA protein to its antibodies in the cocktail to form the HMW-MAA protein-antibody complexes. The HMW-MAA protein-antibody complexes are then detected by commonly used techniques. Detection of the complexes can be facilitated by coupling an antibody to a detectable substance such as an enzyme, prosthetic group, fluorescent material, luminescent material, bioluminescent material, and radioactive material. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, and phycoerythrin; an example of a luminescent material is luminol; examples of bioluminescent materials include luciferase, luciferin, and aequorin; and examples of suitable radioactive material include ¹²⁵I, ¹³¹I, ³⁵S, and ³H.

Flow cytometry and immunohistochemistry are two techniques commonly employed in detecting the HMW-MAA protein on a cell. Flow cytometers are instruments that determine the characteristics of cells in a complex mixture. Cells are led in a stream past an illumination and light detection system. As the cells traverse the illumination spot one by one, a microscope objective collects the scattered and fluorescence light from the cells and directs it to a set of photomultipliers. Temporal, spatial, and chromatic filters eliminate background light and separate the signals from different fluorophores. Digital acquisition electronics measure the intensity of the light pulses from each of the photomultiplier tubes. Immunohistochemistry allows the localization of antigens in tissue sections by the use of labeled antibodies as specific reagents through antigen-antibody interactions that are visualized by a marker described above.

A device of the invention can be used to isolate cells expressing the HMW-MAA protein. Typically, a sample containing cells that express the HMW-MAA protein is provided. In some embodiments, the sample is a body fluid containing circulating cancer cells or a suspension of tumor tissues. The sample is contacted with a device of the invention to allow binding of the HMW-MAA protein to its antibodies. The bound cells (i.e., cells expressing the HMW-MAA protein) are subsequently separated from the unbound components (i.e., cells that do not express the HMW-MAA protein) in the sample by suitable means such as cell sorting, magnetic force, filtration, and centrifugation. Once the bound cells are collected, further analysis of the cells may be performed. For example, the presence of a DNA, mRNA, or protein marker may be determined.

Many cancer diagnostic methods are provided in this invention. These methods can also be used to determine the efficacy of a given treatment regime. One method involves the use of a cocktail of the invention to monitor the HMW-MAA protein levels in tissues and body fluids. In this method, a tissue or body fluid sample from a subject is provided. The tissue is of a type susceptible to cancer or the metastasis of the cancer. The body fluid contains circulating cells. The cancer to be detected is of a type in which the HMW-MAA protein is expressed. The amount of the HMW-MAA protein in the sample is determined with a cocktail of the invention and compared to a control value. If the amount of the HMW-MAA protein in the test sample is higher than a control value, the subject is likely to be suffering from the cancer.

Another method of the invention involves a PE tissue sample from a subject. The tissue is of a type susceptible to cancer or the metastasis of the cancer. The cancer is of a type in which the HMW-MAA gene is expressed. The expression level of the HMW-MAA gene or the methylation level of the HMW-MAA gene promoter in the sample is determined. If the expression level of the HMW-MAA gene in the sample is higher than the control expression level, or if the methylation level of the HMW-MAA gene promoter in the sample is lower than the control methylation level, the subject is likely to be suffering from the cancer.

Still another diagnostic method of the invention involves a body fluid sample from a subject, wherein the sample contains DNA that exists as acellular DNA in the body fluid. The presence or absence of an HMW-MAA genomic sequence in the DNA is determined. If the HMW-MAA genomic sequence is present in the DNA, the subject is likely to be suffering from cancer.

A further diagnostic method of the invention involves a tissue or body fluid sample from a subject. The tissue is of a type susceptible to cancer or the metastasis of the cancer. The body fluid contains cells. The cancer is of a type in which the HMW-MAA gene is expressed. The amount of the HMW-MAA mRNA in the sample is determined and compared with a control value. If the amount of the HMW-MAA mRNA in the sample is higher than the control value, the subject is likely to be suffering from the cancer.

Moreover, the invention provides a method for diagnosing non-lobular breast cancer or pancreatic cancer. A tissue sample or a body fluid sample from a subject is provided. The tissue is of a type susceptible to non-lobular breast cancer or pancreatic cancer or the metastasis of the non-lobular breast cancer or pancreatic cancer. The sample contains cellular DNA. The body fluid contains cells. The expression level of the HMW-MAA gene in the sample or the methylation level of the HMW-MAA gene promoter in the DNA is determined and compared with a control value. If the expression level of the HMW-MAA gene in the sample is higher than a control expression level, or if the methylation level of the HMW-MAA gene promoter in the DNA is lower than a control methylation level, the subject is likely to be suffering from the cancer.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

A “tissue” sample from a subject may be a biopsy specimen sample, a normal or benign tissue sample, a cancer or tumor tissue sample, a freshly prepared tissue sample, a frozen tissue sample, a PE tissue sample, a primary cancer or tumor sample, or a metastasis sample. Exemplary tissues include, but are not limited to, epithelial, connective, muscle, nervous, heart, lung, brain, eye, stomach, spleen, bone, pancreatic, kidney, gastrointestinal, skin, uterus, thymus, lymph node, colon, breast, prostate, ovarian, esophageal, head, neck, rectal, testis, throat, thyroid, intestinal, melanocytic, colorectal, liver, gastric, and bladder tissues. A tissue is “susceptible to cancer or the metastasis of the cancer” if cancer can originate or spread in the tissue.

The term “body fluid” refers to any body fluid in which acellular DNA or cells (e.g., cancer cells) may be present, including, without limitation, blood, serum, plasma, bone marrow, cerebral spinal fluid, peritoneal/pleural fluid, lymph fluid, ascite, serous fluid, sputum, lacrimal fluid, stool, and urine.

“Acellular DNA” refers to DNA that exists outside a cell in a body fluid of a subject or the isolated form of such DNA, while “cellular DNA” refers to DNA that exists within a cell or is isolated from a cell.

Tissue and body fluid samples can be obtained from a subject using any of the methods known in the art. Methods for extracting acellular DNA from body fluid samples are well known in the art. Commonly, acellular DNA in a body fluid sample is separated from cells, precipitated in alcohol, and dissolved in an aqueous solution. Methods for extracting cellular DNA from tissue and body fluid samples are also well known in the art. Typically, cells are lysed with detergents. After cell lysis, proteins are removed from DNA using various proteases. DNA is then extracted with phenol, precipitated in alcohol, and dissolved in an aqueous solution.

The genomic sequence of HMW-MAA is known. The presence of the HMW-MAA genomic sequence or a portion thereof can be determined using many techniques well known in the art. Such techniques include, but are not limited to, Southern blot, sequencing, and PCR.

A “promoter” is a region of DNA extending 150-300 bp upstream from the transcription start site that contains binding sites for RNA polymerase and a number of proteins that regulate the rate of transcription of the adjacent gene. The promoter region of the HMW-MAA gene is well known in the art. Methylation of the HMW-MAA gene promoter can be assessed by any method commonly used in the art, for example, methylation-specific PCR (MSP), bisulfite sequencing, or pyrosequencing.

MSP is a technique whereby DNA is amplified by PCR dependent upon the methylation state of the DNA. See, e.g., U.S. Pat. No. 6,017,704. Determination of the methylation state of a nucleic acid includes amplifying the nucleic acid by means of oligonucleotide primers that distinguish between methylated and unmethylated nucleic acids. MSP can rapidly assess the methylation status of virtually any group of CpG sites within a CpG island, independent of the use of methylation-sensitive restriction enzymes. This assay entails initial modification of DNA by sodium bisulfite, converting all unmethylated, but not methylated, cytosines to uracils, and subsequent amplification with primers specific for methylated versus unmethylated DNA. MSP requires only small quantities of DNA, is sensitive to 0.1% methylated alleles of a given CpG island locus, and can be performed on DNA extracted from body fluid, tissue, and PE samples. MSP eliminates the false positive results inherent to previous PCR-based approaches which relied on differential restriction enzyme cleavage to distinguish methylated from unmethylated DNA. This method is very simple and can be used on small amounts of tissue or few cells and fresh, frozen, or PE sections. MSP product can be detected by gel electrophoresis, CAE (capillary array electrophoresis), or real-time quantitative PCR.

Bisulfite sequencing is widely used to detect 5-MeC (5-methylcytosine) in DNA, and provides a reliable way of detecting any methylated cytosine at single-molecule resolution in any sequence context. The process of bisulfite treatment exploits the different sensitivity of cytosine and 5-MeC to deamination by bisulfite under acidic conditions, in which cytosine undergoes conversion to uracil while 5-MeC remains unreactive.

A “control methylation lever” may be the methylation level of the HMW-MAA gene promoter in a normal DNA from a normal tissue or cells in a body fluid of a normal subject, or the methylation level of the HMW-MAA gene promoter in a normal DNA from a normal tissue of a test subject. Preferably, the normal tissue is obtained from a site where the cancer being tested for can originate or metastasize. By “normal” is meant without cancer.

“Gene expression” is a process by which a gene is transcribed into an mRNA, which in turn is translated into a protein. The expression level of the HMW-MAA gene can be measured, e.g., by the amount of the HMW-MAA mRNA, the amount of the HMW-MAA protein, or a combination thereof. The expression level of the HMW-MAA gene may be reduced, e.g., by inhibiting the transcription from DNA to mRNA or the translation from mRNA to protein. Alternatively, the expression level of the HMW-MAA gene may be reduced by preventing mRNA or protein from performing their normal functions. For example, the mRNA may be degraded through anti-sense RNA, ribozyme, or siRNA; the protein may be blocked by an antibody.

Gene expression can be detected and quantified at mRNA or protein level using a number of means well known in the art. To measure mRNA levels, cells in biological samples (e.g., cultured cells, tissues, and body fluids) can be lysed and the mRNA levels in the lysates or in RNA purified or semi-purified from the lysates determined by any of a variety of methods familiar to those in the art. Such methods include, without limitation, hybridization assays using detectably labeled gene-specific DNA or RNA probes and quantitative or semi-quantitative real-time RT-PCR methodologies using appropriate gene-specific oligonucleotide primers. Alternatively, quantitative or semi-quantitative in situ hybridization assays can be carried out using, for example, unlysed tissues or cell suspensions, and detectably (e.g., fluorescently or enzyme-) labeled DNA or RNA probes. Additional methods for quantifying mRNA levels include RNA protection assay (RPA), cDNA and oligonucleotide microarrays, and calorimetric probe based assays.

Methods of measuring protein levels in biological samples are also known in the art. Many such methods employ antibodies (e.g., monoclonal or polyclonal antibodies) that bind specifically to target proteins. In such assays, an antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein-measuring assays (e.g., ELISA or Western blot) can be applied to body fluids or to lysates of test cells, and others (e.g., immunohistological methods or fluorescence flow cytometry) applied to unlysed tissues or cell suspensions. Methods of measuring the amount of a label depend on the nature of the label and are known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

In some embodiments, the expression level of the HMW-MAA gene is determined by detecting the HMW-HAA mRNA using qRT or by detecting the HMW-MAA protein using an antibody to the HMW-MAA protein or a cocktail of the invention. In some embodiments, the amount of the HMW-HAA mRNA and the amount of the HMW-MAA protein are combined in determining the expression level of the HMW-MAA gene or whether a subject is likely to be suffering from cancer.

A “control expression level” may be the amount of the HMW-MAA mRNA or protein in a normal tissue or body fluid of a normal subject, or the amount of the HMW-MAA mRNA or protein in a normal tissue of a test subject.

As used herein, “cancer” refers to a disease or disorder characterized by uncontrolled division of cells and the ability of these cells to spread, either by direct growth into adjacent tissue through invasion, or by implantation into distant sites by metastasis. Exemplary cancers include, but are not limited to, primary cancer, metastatic cancer, AJCC stage I, II, III, or IV cancer, carcinoma, lymphoma, leukemia, sarcoma, mesothelioma, glioma, germinoma, choriocarcinoma, prostate cancer, lung cancer, breast cancer (including lobular, non-lobular, ductal, non-ductal, invasive, and non-invasive), colorectal cancer, gastrointestinal cancer, bladder cancer, pancreatic cancer, endometrial cancer, ovarian cancer, melanoma, brain cancer, testicular cancer, kidney cancer, skin cancer, thyroid cancer, head and neck cancer, liver cancer, esophageal cancer, gastric cancer, intestinal cancer, colon cancer, rectal cancer, myeloma, neuroblastoma, and retinoblastoma. Preferably, the cancer is a cancer where the HMW-MAA gene is expressed, such as melanoma, breast cancer, brain cancer, lung cancer, gastrointestinal cancer, sarcoma, and pancreatic cancer.

The discovery that the HMW-MAA gene is expressed in non-lobular breast cancer and pancreatic cancer cells is useful for identifying compounds for treating non-lobular breast cancer and pancreatic cancer. For example, a non-lobular breast cancer or pancreatic cancer cell may be contacted with a test compound. The expression levels of the HMW-MAA gene in the cell prior to and after the contacting step are compared. If the expression level of the HMW-MAA gene in the cell decreases after the contacting step, the test compound is identified as a candidate compound for treating non-lobular breast cancer and pancreatic cancer.

Similarly, a subject suffering from non-lobular breast cancer or pancreatic cancer may be contacted with a test compound. Samples of cancer tissues or body fluids containing cancer cells are obtained from the subject. The expression level of the HMW-MAA gene in a sample obtained from the subject prior to the contacting step is compared with the expression level of the HMW-MAA gene in a sample obtained from the subject after the contacting step. If the expression level of the HMW-MAA gene decreases after the contacting step, the test compound is identified as a candidate compound for treating non-lobular breast cancer and pancreatic cancer.

The test compounds of the present invention can be obtained using any of the numerous approaches (e.g., combinatorial library methods) known in the art. See, e.g., U.S. Pat. No. 6,462,187. Such libraries include, without limitation, peptide libraries, peptoid libraries (libraries of molecules having the functionalities of peptides, but with a novel, non-peptide backbone that is resistant to enzymatic degradation), spatially addressable parallel solid phase or solution phase libraries, synthetic libraries obtained by deconvolution or affinity chromatography selection, and the “one-bead one-compound” libraries. Compounds in the last three libraries can be peptides, non-peptide oligomers, or small molecules. Examples of methods for synthesizing molecular libraries can be found in the art. Libraries of compounds may be presented in solution, or on beads, chips, bacteria, spores, plasmids, or phages.

The candidate compounds so identified, as well as compounds known to reduce the expression level of the HMW-MAA gene in a cell or subject, can be used to reduce the expression of the HMW-MAA gene in non-lobular breast cancer and pancreatic cancer cells in vitro and in vivo. Compounds known to reduce the expression level of the HMW-MAA gene in a cell or subject include HMW-MAA mimics (17, 18: U.S. Pat. No. 5,780,029) and HMW-MAA-specific monoclonal antibody (19).

In one embodiment, the method involves contacting a non-lobular breast cancer or pancreatic cancer cell with an agent that reduces the expression level of the HMW-MAA gene in the cell. To treat a subject suffering from non-lobular breast cancer or pancreatic cancer, an effective amount of an agent that reduces the expression level of the HMW-MAA gene is administered to the subject. A subject to be treated may be identified in the judgment of the subject or a health care professional, and can be subjective (e.g., opinion) or objective (e.g., measurable by a test or diagnostic method such as those described above).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect).

In some embodiments, a non-lobular breast cancer or pancreatic cancer cell or a subject suffering from non-lobular breast cancer or pancreatic cancer is further treated with other compounds or radiotherapy.

In some embodiments, polynucleotides (i.e., antisense nucleic acid molecules, ribozymes, and siRNAs) are administered to a subject. Polynucleotides can be delivered to target cells by, for example, the use of polymeric, biodegradable microparticle or microcapsule devices known in the art. Another way to achieve uptake of the nucleic acid is using liposomes, prepared by standard methods. The polynucleotides can be incorporated alone into these delivery vehicles or co-incorporated with tissue-specific or tumor-specific antibodies. Alternatively, one can prepare a molecular conjugate composed of a polynucleotide attached to poly-L-lysine by electrostatic or covalent forces. Poly-L-lysine binds to a ligand that can bind to a receptor on target cells. “Naked DNA” (i.e., without a delivery vehicle) can also be delivered to an intramuscular, intradermal, or subcutaneous site. A preferred dosage for administration of polynucleotide is from approximately 10⁶ to 10¹² copies of the polynucleotide molecule.

For treatment of cancer, a compound is preferably delivered directly to tumor cells, e.g., to a tumor or a tumor bed following surgical excision of the tumor, in order to treat any remaining tumor cells. For prevention of cancer invasion and metastases, the compound can be administered to, for example, a subject that has not yet developed detectable invasion and metastases but is found to have increased expression level of the HMW-MAA gene.

The compounds of the invention can be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

In one embodiment, the compounds are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Example I

### Human High Molecular Weight-Melanoma Associated Antigen (HMW-MAA) is Expressed in Breast Cancer

### Introduction

Breast cancer is the most commonly identified and one of the deadliest neoplasms afflicting women in Western countries. The recent trend toward improvement of the mortality of rate breast cancer is largely due to increased diagnosis of early stage disease, while therapeutic options for advanced stage breast carcinomas are still fairly limited. Thus, there is a need to better understand the molecular basis of breast cancer initiation and progression and to use this knowledge for the design of targeted, molecular-based therapies or application of other novel strategies for the treatment of breast cancer patients.

Recently, the promoter region DNA methylation of HMW-MAA was reported to play a critical role in regulating the level of HMW-MAA expression both in melanoma cell lines and in surgically removed tumors (20). The major objective of this study was to determine whether ductal carcinoma of the breast expressed HMW-MAA or not, to assess the mechanisms regulating the expression of HMW-MAA in breast cancer, and to discuss practical applications for use of HMW-MAA in immunodiagnostics, as well as in the application of immunotherapies or molecular-based therapies for the treatment of patients with breast cancer.

### Materials and Methods

### Cell Lines

Six established breast cancer cell lines (T-47D, MCF-7, MDA-MB435S, 734B, ZR-75-1, MDA-MB231) from ATCC (Manassas, Va.) were analyzed in this study. Additionally, 13 melanoma cell lines (MA-MM) established at John Wayne Cancer Institute (JWCI), 2 established colorectal cancer cell lines, SW480 and DLD-1, from ATCC, and 2 established gastric cancer cell lines, MKN1 and MKN28, from RIKEN BRC (Ibaraki, Japan) were assessed. Genomic DNA was extracted from cells, as previously described (21). Total RNA was extracted using TRI Reagent (Molecular Research Center, Inc., Cincinnati, Ohio), according to the manufacturer's protocol. Quality and quantity of extracted DNA and total RNA were measured by LTV absorption spectrophotometry.

For HMW-MAA gene expression studies, T-47D, MCF-7, MDA-MB435S, and ZR-75-1 were treated with 5-aza-2-deoxycytidine (5Aza, Sigma Chemical Co., St. Louis, Mo.), a known inhibitor of methylation, and with Trichostatin A (TSA, Wako Biochemicals, Osaka, Japan), a histone deacetylation (HDAC) inhibitor, as previously described (22).

### Human Breast Tissues

Paraffin-embedded (PE) primary tissues from breast cancer patients and PE normal breast tissues from non-malignant breast tumor patients treated by JWCI physicians were obtained from the Division of Surgical Pathology, Saint John's Health Center (SJHC). Informed consents were obtained from patients for the use of all specimens and human subject approval was granted from the JWCI/SJHC joint Institutional Review Board prior to beginning the study. All primary tumors were assessed by hematoxylin & eosin (A&E) and immunohistochemistry (IHC) staining.

### DNA and RNA Isolation

Several 5 μm sections were cut with a microtome from PE blocks under sterile conditions, as described previously (23). One section for each tumor was stained with H&E after deparaffinization as references of microdissection. For DNA methylation analysis, the tumors were precisely microdissected under a microscope from one section as previously described (24) and subsequently digested with 50 μl of proteinase K containing lysis buffer. For analysis of mRNA expression level, the tumors were also precisely microdissected under a microscope from two sections and digested with 50 μl of proteinase K containing lysis buffer, and subsequently RNA was extracted with RNAwiz RNA Isolation Kit (Ambion, Austin, Tex.) following the manufacturer's protocol. RNA extraction was performed in a designated sterile laminar flow hood using RNase/DNase-free plasticware. Pellet Paint (Novagen, Madison, Wis.) was used in the precipitation procedure to enhance the recovery of RNA. The RNA was quantified and assessed for purity using UV spectrophotometry and the RIBOGreen detection assay (Molecular Probes, Eugene, Oreg.). The expression of mRNA for glyceraldyhyde-3-phosphate dehydrogenase (GAPDH), an internal reference housekeeping gene, was assessed by reverse transcription (RT-PCR) to verify the integrity of the all RNA samples. Specimens with undetectable or low GAPDH mRNA expression were not used for subsequent analysis. Tissue processing, RNA extraction, and a quantitative real-time reverse-transcription PCR (qRT) assay set-up were performed in separately designated rooms to prevent cross-contamination, as described previously (25).

Analysis of mRNA Expression Level

Reverse transcriptase reactions were performed using Moloney murine leukemia virus reverse transcriptase (Probega, Madison, Wis.) with oligo-dT primer (25). For clinical specimens, random primers were additionally used. The qRT assay was performed using iCycler iQ RealTime Thermocycler Detection system (Bio-Rad Laboratories, Hercules, Calif.); cDNA from 250 ng of total RNA was used for each reaction (25). The PCR reaction mixture consisted of 0.2 uM of each primer, 0.5 uM FRET probe, 1 U of AmpliTaq Gold polymerase (Applied Biosystems, Branchburg, N.J.), 200 uM of each deoxynucleoside triphosphate, 4.5 mM MgCl₂, and PCR buffer to a final volume of 25 ul. To avoid possible amplification of contaminating genomic DNA, primers were designed so that each PCR product overlapped at least one exon-exon junction, as previously described (25). The primer and probe sequences used were as follows: HMW-MAA, 5′-TGGAAGAACAAAGGTCTCTGG-3′ (forward), 5′-GCTGGCCAAGAGATTGGAG-3′ (reverse), 5′-FAM-AGGATCACCGTGGCTGCTCT-BHQ-1-3′ (FRET probe); GAPDH, 5′-GGGTGTGAACCATGAGAAGT-3′ (forward), 5′-GACTGTGGTCATGAGTCCT-3) (reverse), and 5′-FAM-CAGCAATGCCTCCTGCACCACCAA-BHQ-1-3′ (FRET probe). Samples were amplified with a precycling hold at 95° C. for 10 min, followed by 45 cycles of denaturation at 95° C. for 1 min, annealing at 63° C. for 1 min for HMW-MAA and annealing at 55° C. for 1 min for GAPDH, extension at 72° C. for 1 min, and final hold at 72° C. for 7 min. Plasmids for individual gene cDNA were constructed as described previously (25). The standard curve was generated by using a threshold cycle (Ct) of nine serially diluted (10 to 10⁸ copies) plasmids containing HMW-MAA and GAPDH cDNA. The Ct of each sample was interpolated from the standard curve, and the number of mRNA copies was calculated by the iCycler iQ RealTime Detection System software (Bio-Rad Laboratories), as previously described (25). Established melanoma cell lines were used as positive controls. Reagent controls for qRT assays were included in each assay, as described previously (25). Each assay was repeated in duplicate to verify the results. The mean mRNA copy number was used for subsequent statistical analysis.

Monoclonal Antibodies (mAb) and Flow Cytometry

The mouse anti-HMW-MAA mAbs (225.28, 763.74, VT80.12, VF4-TP108, VF1-TP41.2, VF20-VT5.1, TP61.5) have been described previously (1). Cells (1×10⁶) were incubated at 4° C. for 1 h with each HMW-MAA-specific mAb (1 μg) or an isotype-matched control antibody, washed twice with PBS/0.5% BSA, and incubated at 4° C. for an additional 30 min with an optimal amount of RPE-labeled F(ab′)₂ fragments of goat anti-mouse IgG (Santa Cruz Biotechnology, Santa Cruz, Calif.). The cells were then washed twice, fixed in 4% paraformaldehyde, and analyzed by flow cytometry (FACSCalibur, Becton Dickinson, Mountainview, Calif.). Cells (1×10⁴) were acquired for each sample. Debris, cell clusters, and dead cells were gated out by light-scattered assessment before single parameter histograms were drawn. Data were analyzed with Cell Quest software (Becton Dickinson).

### Immunohistochemistry

Expression of HMW-MAA in cell lines was assessed by IHC. Cells were cultured on Lab-Tek II Chamber slides (Nalge Nunc International, Naperville, Ill.). Specimens were fixed in 4% paraformaldehyde and then incubated overnight with cocktailed HMW-MAA mAb (1:100 dilution) at 4° C. Negative control cells were treated with non-immunized immunoglobulin fraction under equivalent conditions and with no primary antibody. For the secondary developing reagents, LSAB+kit (DAB) (Dako Corp., Carpinteria, Calif.) was used. Slides were counterstained with H&E for reading. Expression of HMW-MAA in tissue was also assessed by IHC. Five μm sections were deparafinized in xylene and slides were bathed in 1 mM EDTA and boiled for 15 min. The sections were incubated with cocktailed HMW-MAA mAb at a dilution of 1:100 and kept at 4° C. overnight. For the secondary developing reagents, CSAII, Biotin-Free Catalyzed Signal Amplification System (Dako) was used following the manufacturer's protocol. Slides were developed with Vector VIP Peroxidase Substrate Kit (Vector Laboratories, Burlingame, Calif.). Slides were counterstained with H&E for reading.

### Detection of Hypermethylation

Sodium bisulfite modification (SBM) was applied on extracted genomic DNA of tissue specimens and cell lines for methylation-specific PCR (MSP) (21. Methylation-specific and unmethylation-specific primer sets were designed; optimization for MSP included annealing temperature, Mg²⁺ concentration, and cycle number for specific amplification of the methylated and unmethylated target sequences. The primers were dye-labeled for automatic detection in capillary array electrophoresis (CAE). The methylation-specific primer set was as follows: forward, 5′-D4-AGTTTAAGTTTGAAATTCGAGCG-3; and reverse, 5′-AAACTAAATAAAACGAACGCGA-3′. The unmethylation-specific primer set was as follows: forward, 5′-D3-GGAGTTTAAGTTTGAAATTTGAGTG-3′; and reverse, 5′-CTAAAAACTAAAAACTAAATAAAACAAACACA-3′; PCR amplification was done in a 10 μL reaction volume with 1 μL template for 36 cycles of 30 seconds at 94° C., 30 seconds at 63° C. for methylation and 60° C. for unmethylation, and 30 seconds at 72° C., followed by a 7-minute final extension at 72° C. The PCR reaction mixture consisted of 0.3 μM of each primer, 1 U of AmpliTaq Gold polymerase (Applied Biosystems), 200 μM of each deoxynucleoside triphosphate, 2.5 mM MgCl², and PCR buffer to a final volume of 10 μl. A universal unmethylated control was synthesized from normal DNA by phi-29 DNA polymerase and served as a positive unmethylated control (26). Unmodified lymphocyte DNA was used as a negative control for methylated and unmethylated reactions. Sssl Methylase-(New England Bio Labs, Beverly, Mass.) treated lymphocyte DNA was used as a positive methylated control. PCR products were detected and analyzed by CAE (CEQ 8000XL; Beckman Coulter, Inc., Fullerton, Calif.) with CEQ 8000 software version 8.0 (Beckman Coulter) as described previously (24). Methylation status was determined by the ratio of the signal intensities of methylated and unmethylated PCR products; samples with methylated to unmethylated ratio larger than 0.1 were determined to be methylated.

### Statistical Analysis

Statistical analysis of the data was performed using the unpaired Student's t test and Mann-Whitney U test. P values were two-sided where a value of <0.05 was considered statistically significant.

### Results

HMW-MAA mRNA Expression in Cell Lines

The expression of HMW-MAA mRNA in melanoma, breast cancer, gastric cancer, colon cancer cell lines, and normal healthy donor PBL was initially assessed by RT-PCR. The frequency of HMW-MAA mRNA expression was 100% (9 of 9) of melanoma cell lines, 83.3% (5 of 6) of breast cancer cell lines, 0% (0 of 2) of colon cancer cell lines, 0% (0 of 4) of gastric cancer cell lines, and 0% (0 of 7) normal healthy donor PBL. In addition, HMW-MAA mRNA expression level in 13 melanoma cell lines, 6 breast cancer cell lines, 4 gastric cancer cell lines, 2 colon cancer cell lines, and 7 normal healthy donor PBL samples was assessed by a qRT assay. Breast cancer cell lines showed high HMW-MAA expression level, as did melanoma cell lines. This finding demonstrated the expression of HMW-MAA mRNA levels by breast cancer cell lines.

Recently, the promoter DNA methylation of HMW-MAA was reported to play a critical role in regulating the level of HMW-MAA expression both melanoma cell lines and in surgically removed tumors (20). DNA methylation of the HMW-MAA CpG island promoter region was assessed in 4 breast cancer cell lines by the MSP assay. Among the four breast cancer cell lines studied, two cell lines (MCF-7 and ZR75-1) were fully methylated and the other two cell lines (MDA-MB435 and T47-D) were hypomethylated. The correlation between HMW-MAA mRNA expression and DNA methylation of the HMW-MAA CpG island promoter region was assessed. The HMW-MAA mRNA expression of hypermethylated cell lines was lower than that of hypomethylated cell lines. These results demonstrate that promoter DNA methylation of HMW-MAA regulates the mRNA expression of HMW-MAA in breast cancer.

The expression of HMW-MAA protein in MDA-MB435 was examined by flow cytometric analysis with each HMW-MAA specific mAb (225.28, 763.74, VT80.12, VF4-TP108, VF1-TP41.2, VF20-VT5.1, TP61.5). HMW-MAA was expressed in MDA-MB435 by all HMW-MAA specific mAbs, even though there was a small difference in expression level among those mAbs. A cocktail of five HMW-MAA specific mAbs was used for the IHC study, because some mAbs demonstrated higher specificity and sensitivity by flow cytometric analysis than mAb 225.28 and mAb 763.74, which have been reported to be effective in previous HMW-MAA IHC studies. The correlation between HMW-MAA DNA promoter region methylation and protein expression was assessed by IHC. MDA-MB435 (hypomethylated) and T47-D (hypomethylated) breast cancer cell lines were stained by IHC, but MCF-7 (hypermethylated) and ZR75-1 (hypermethylated) were unstained. These results demonstrate that promoter DNA methylation of HMW-MAA plays an important role in regulating the protein level of HMW-MAA expression in breast cancer, as well as melanoma, in vitro.

To determine if cells with hypermethylated HMW-MAA can be induced to increase expression HMW-MAA mRNA, breast cancer cell lines (MCF-7 and ZR75-1) were treated with 5-Aza and TSA. The HMW-MAA mRNA copy number was increased after treatment with 5-Aza and TSA alone in hypermethylated cell lines. These results suggest that TSA treatment induce upregulation of HMW-MAA gene expression in hypermethylated cell lines.

HMW-MAA mRNA Expression in Tissues

Next, tissue samples were examined to confirm the results found in the breast cancer cell lines. HMW-MAA mRNA expression in primary breast cancer tissues and non-malignant breast tissue was first assessed. Sixty-nine primary breast cancer tissues from 55 breast cancer patients and 23 normal breast tissues from 23 non-malignant breast tumor patients were assessed. The mRNA copy ratio of HMW-MAA/GAPDH varied from 0.000083 to 0.863 (mean±S.E., 0.22±0.02) in primary breast cancer and from 0.0274 to 0.3 (mean±S.E., 0.10±0.07) in normal breast tissues. The mean HMW-MAA mRNA copy ratio in breast cancer patients was significantly higher than in normal breast tissues from the non-malignant breast tumor patients (p=0.0036). Primary breast cancer samples were classified as T1 (n=38) or T2 (n=29) by tumor size. There was no difference in HMW-MAA mRNA expression between the T1 and T2 groups, but the HMW-MAA expressions of T1 (p=0.0081) and T2 (p=0.0044) were significantly higher than that of normal breast tissues, respectively.

Twenty tissue samples of primary breast cancer were assessed by MSP, and one out of 20 (5%) was methylated. HMW-MAA mRNA expression of hypermethylated samples was low compared to hypomethylated samples. These findings suggest that there may be a correlation between HMW-MAA mRNA expression and DNA methylation of the HMW-MAA CpG island promoter region in vivo.

### Discussion

HMW-MAA is a melanoma marker of particular interest since 1) it is highly expressed at the surface of melanoma cells, 2) it has restricted distribution in normal tissues (20, 27) (Ferrone S 1993), 3) the induction of specific humoral response to anti-idiotypic anti-HMW-MAA mAb increases survival in patients with advanced melanoma (Ferrone S, 1993; (17), and 4) it plays a critical role in tumor growth and metastasis (9, 11, 17). Despite the biological importance of HMW-MAA in melanoma, to date there have been few studies of HMW-MAA in other malignant tumors or cancers.

First, the mRNA expression of HMW-MAA in breast cancer, gastric cancer and colon cancer cell lines, as well as melanoma, was assessed. Breast cancer cell lines showed high mRNA expressions of HMW-MAA compared to gastric cancer, colon cancer, and PBL. These findings suggest that HMW-MAA mRNA is expressed in breast cancer cell lines as well as melanoma cell lines.

The HMW-MAA is highly immunogenic in BALB/c mice, as indicated by the high frequency of HMW-MAA-specific antibody-secreting hybridomas generated from BALB/c mice immunized with HMW-MAA-bearing human melanoma cells. As a result, a large number of mouse anti-HMW-MAA mAb have been developed (Michael R C, 2004). To date, mAb 763.74 and mAb 225.28 have been mainly used as HMW-MAA/mAb in published papers (7, 15, 20). Whether breast cancer would express HMW-MAA protein corresponding to HMW-MAA mRNA levels, and, subsequently, which HMW-MAA mAb should be used were next examined. The results demonstrated that breast cancer showed high expression of each of the 7 HMW-MAA mAbs by flow cytometry. Therefore, it was decided to use cocktailed HMW-MAA mAbs for IHC study. 5 cocktailed HMW-MAA mAbs (225.28, 763.74, VF4-TP108, VF1-TP41.2, TP61.5) were used for cell lines, and 3 cocktailed HMW-MAA mAbs (763.74, VT80.12, VF20-VT5-1) for PE tissues. The breast cancer cell line was stained by cocktailed HMW-MAA mAbs.

Recently, the promoter region DNA methylation of HMW-MAA was reported to play a critical role in regulating the level of HMW-MAA expression in melanoma cell lines (20). That promoter region DNA methylation also regulates HMW-MAA expression in breast cancer cell lines was hypothesized. The results demonstrated that the HMW-MAA mRNA expression of hypermethylated breast cancer cell lines was lower than that of hypomethylated lines. In addition, HMW-MAA was stained by IHC in hypomethylated but not hypermethylated breast cancer cell lines. Promoter region DNA methylation is correlated with HMW-MAA mRNA expression and protein expression in breast cancer cell lines. These findings support our hypothesis that HMW-MAA gene can be inactivated by promoter region hypermethylation. Previously, the restoration of gene expression by treatment with the demethylating agent 5-Aza had been demonstrated in melanoma cell lines as a confirmation of the inactivating mechanism (20). This study showed that HMW-MAA mRNA expression in breast cancer cell lines was upregulated after treatment with 5-Aza and TSA alone or in combination, expect for cell line MCF-7. The results suggested that HMW-MAA expression is activated not only by DNA demethylation, but also histone deacethylase inhibition in breast cancer cell lines.

HMW-MAA expression and methylation in breast cancer tissue specimens were also analyzed. The findings demonstrated that HMW-MAA mRNA was expressed significantly higher in primary breast cancer tissue than in non-malignant breast tissue by a qRT assay, and HMW-MAA was also expressed in primary breast cancer by IHC. These results suggest that HMW-MAA may be a valuable marker for breast cancer.

In addition, hypomethylated primary breast cancers showed higher expression of HMW-MAA mRNA compared to hypermethylated primary breast cancers. These observations suggest that DNA methylation may serve as a common mechanism for tumor antigen gene expression control in breast cancer tissues.

The results also showed that there is no difference in HMW-MAA expression between T1 and T2 primary breast cancers. There may be no correlation between HMW-MAA expression and tumor progression in primary breast cancer. HMW-MAA expression may start in the early stages of breast cancer.

The results of this study have implications for the development of therapeutic strategies that specifically target HMW-MAA.

### REFERENCES

1. Campoli M R, Chang C C, Kageshita T, Wang X, McCarthy J B, Ferrone S. Human high molecular weight-melanoma-associated antigen (HMW-MAA): a melanoma cell surface chondroitin sulfate proteoglycan (MSCP) with biological and clinical significance. Crit Rev Immunol 2004, 24:267-96.
2. Kageshita T, Johno M, Ono T, Arao T, Imai K. Immunohistochemical analysis of antimelanoma monoclonal antibodies, with special reference to fetal tissue distribution. J Invest Dermatol 1985, 85:535-7.
3. Chekenya M, Rooprai H K, Davies D, Levine J M, Butt A M, Pilkington G J. The NG2 chondroitin sulfate proteoglycan: role in malignant progression of human brain tumours. Int J Dev Neurosci 1999, 17:421-35.
4. Chekenya M, Pilkington G J. NG2 precursor cells in neoplasia: functional, histogenesis and therapeutic implications for malignant brain tumours. J Neurocytol 2002, 31:507-21.
5. Shoshan Y, Nishiyama A, Chang A, Mork S, Barnett G H, Cowell J K, et al. Expression of oligodendrocyte progenitor cell antigens by gliomas: implications for the histogenesis of brain tumors. Proc Natl Acad Sci USA 1999, 96: 10361-6.
6. Godal A, Bruland O, Haug E, Aas M, Fodstad O. Unexpected expression of the 250 kD melanoma-associated antigen in human sarcoma cells. Br J Cancer 1986, 53:839-41.
7. Dell'Erba L, Calo-Gabrieli G, Caruso M L, Thomas R, Cortino G, Valentini A M, et al. Immunohistochemical reactivity of anti-melanoma monoclonal antibody 225.28S in human breast cancer biopsies. Anticancer Res 2001, 21:925-30.
8. Schlingemann R O, Rietveld F J, Kwaspen F, van de Kerkhof P C, de Waal R M, Ruiter D J. Differential expression of markers for endothelial cells, pericytes, and basal lamina in the microvasculature of tumors and granulation tissue. Am J Pathol 1991, 138:1335-47.
9. Burg M A, Grako K A, Stallcup W B. Expression of the NG2 proteoglycan enhances the growth and metastatic properties of melanoma cells. J Cell Physiol 1998, 177:299-312.
10. Fang X, Burg M A, Barritt D, Dahlin-Huppe K, Nishiyama A, Stallcup W B. Cytoskeletal reorganization induced by engagement of the NG2 proteoglycan leads to cell spreading and migration. Mol Biol Cell 1999, 10:3373-87.
11. Iida J, Meijne A M, Knutson J R, Furcht L T, McCarthy J B. Cell surface chondroitin sulfate proteoglycans in tumor cell adhesion, motility and invasion. Semin Cancer Biol 1996, 7:155-62.
12. Iida J, Pei D, Kang T, Simpson M A, Herlyn M, Furcht L T, McCarthy J B. Melanoma chondroitin sulfate proteoglycan regulates matrix metalloproteinase-dependent human melanoma invasion into type I collagen. J Biol Chem 2001, 276: 18786-94.
13. Eisenmann K M, McCarthy J B, Simpson M A, Keely P J, Guan J L, Tachibana K, et al. Melanoma chondroitin sulphate proteoglycan regulates cell spreading through Cdc42, Ack-1 and p130cas. Nat Cell Biol 1999, 1:507-13.
14. Yang J, Price M A, Neudauer C L, Wilson C, Ferrone S, Xia H, et al. Melanoma chondroitin sulfate proteoglycan enhances FAK and ERK activation by distinct mechanisms. J Cell Biol 2004, 165:881-91.
15. Kageshita T, Nakamura T, Yamada M, Kuriya N, Arao T, Ferrone S. Differential expression of melanoma associated antigens in acral lentiginous melanoma and in nodular melanoma lesions. Cancer Res 1991, 51:1726-32.
16. Kageshita T, Kuriya N, Ono T, Horikoshi T, Takahashi M, Wong G Y, Ferrone S. Association of high molecular weight melanoma-associated antigen expression in primary acral lentiginous melanoma lesions with poor prognosis. Cancer Res 1993, 53:2830-3.
17. Mittelman A, Chen Z J, Yang H, Wong G Y, Ferrone S. Human high molecular weight melanoma-associated antigen (HMW-MAA) mimicry by mouse anti-idiotypic monoclonal antibody MK2-23: induction of humoral anti-HMW-MAA immunity and prolongation of survival in patients with stage 1V melanoma. Proc Natl Acad Sci USA 1992, 89:466-70.
18. Mittelman A, Chen Z J, Liu C C, Hirai S, Ferrone S. Kinetics of the immune response and regression of metastatic lesions following development of humoral anti-high molecular weight-melanoma associated antigen immunity in three patients with advanced malignant melanoma immunized with mouse antiidiotypic monoclonal antibody MK2-23. Cancer Res 1994, 54:415-21.
19. Hafner C, Breiteneder H, Ferrone S, Thallinger C, Wagner S, Schmidt W M, et al. Suppression of human melanoma tumor growth in SCID mice by a human high molecular weight-melanoma associated antigen (HMW-MAA) specific monoclonal antibody. Int J Cancer 2005, 114:426-32.
20. Luo W, Wang X, Kageshita T, Wakasugi S, Karpf A R, Ferrone S. Regulation of high molecular weight-melanoma associated antigen (HMW-MAA) gene expression by promoter DNA methylation in human melanoma cells. Oncogene 2006, 25:2873-84.
21. Spugnardi M, Tommasi S, Dammann R, Pfeifer G P, Hoon D S. Epigenetic inactivation of RAS association domain family protein 1 (RASSF1A) in malignant cutaneous melanoma. Cancer Res 2003, 63:1639-43.
22. Mori T, Kim J, Yamano T, Takeuchi H, Huang S, Umetani N, et al. Epigenetic up-regulation of C-C chemokine receptor 7 and C-X-C chemokine receptor 4 expression in melanoma cells. Cancer Res 2005, 65:1800-7.
23. Shinozaki M, Fujimoto A, Morton D L, Hoon D S. Incidence of BRAF oncogene mutation and clinical relevance for primary cutaneous melanomas. Clin Cancer Res 2004, 10:1753-7.
24. Umetani N, Takeuchi H, Fujimoto A, Shinozaki M, Bilchik A J, Hoon D S. Epigenetic inactivation of ID4 in colorectal carcinomas correlates with poor differentiation and unfavorable prognosis. Clin Cancer Res 2004, 10:7475-83.
25. Takeuchi H, Fujimoto A, Tanaka M, Yamano T, Hsuch E, Hoon D S. CCL21 chemokine regulates chemokine receptor CCR7 bearing malignant melanoma cells. Clin Cancer Res 2004, 10:2351-8.
26. Umetani N, de Maat M F, Mori T, Takeuchi H, Hoon D S. Synthesis of universal unmethylated control DNA by nested whole genome amplification with phi29 DNA polymerase. Biochem Biophys Res Commun 2005, 329:219-23.
27. Wilson B S, Imai K, Natali P G, Ferrone S. Distribution and molecular characterization of a cell-surface and a cytoplasmic antigen detectable in human melanoma cells with monoclonal antibodies. Int J Cancer 1981, 28:293-300.

### Example II

### Detection of Melanoma Sentinel Lymph Node Metastases by Human High Molecular Weight-Melanoma Associated Antigen

### Abstract

Background: Sentinel lymph node (SLN) biopsy is effective for identifying early stages of metastasis in regional lymph node (LN) metastases in melanoma patients. S-100-, HMB-45-, and MART-1-specific monoclonal antibodies (mAb) are routinely used in immunohistochemistry (IHC) to identify LN micrometastases; however, they have limited specificity and variable sensitivity. There is a need to identify more sensitive and specific IHC biomarkers to increase the accuracy of SLN metastasis detection.

Materials & Methods: LN metastasis (n=84) was investigated by IHC staining of paraffin-embedded archival tissue (PEAT) SLN macrometastases (n=52) and micrometastases (n=32) and normal LNs (n=16) with a three-mAb cocktail that recognize distinct determinants of High Molecular Weight-Melanoma Associated Antigen (HMW-MAA). A quantitative real-time reverse-transcriptase PCR (qRT) was demonstrated to detect and validated HMW-MAA in PEAT metastatic SLNs.

Results: The frequency of HMW-MAA protein expression and staining intensity were significantly higher than MART-1 in both LN macrometastases (P<0.0001 and P<0.0001, respectively) and SLN micrometastases (P<0.0001 and P=0.004, respectively). Specifically, all 52 (100%) LN macrometastases were stained by HMW-MAA mAbs, whereas only 43 specimens (83%) were stained by MART-1 mAb. Furthermore, all 23 (100%) SLN micrometastases were stained by HMW-MAA mAb; only 21 (91%), and 18 (78%) lesions were stained by S-100 and HMB-45 mAb, respectively. HMW-MAA mRNA was detected in 32 of 48 (67%) LN metastases.

Conclusions: The HMW-MAA mAb cocktail is useful to detect melanoma SLN metastasis by IHC staining. In addition, qRT assessment of HMW-MAA mRNA in PE SLN can detect SLN melanoma metastasis. HMW-MAA has utility as a more sensitive and specific biomarker than current common biomarkers, and the use of HMW-MAA can improve occult tumor cell detection via IHC and qRT in SLNs of melanoma.

### Introduction

The most frequent melanoma metastasis site is the regional tumor-draining lymph node (LN) basin. Because the sentinel LN (SLN) represents the first LN in the regional lymphatic basin to receive drainage from the primary tumor, it is likely to be the initial site of early LN metastases. Sentinel lymphadenectomy (SLND), a less invasive method to assess the tumor-draining LN basin, has revolutionized the surgical management of primary malignant melanoma.¹⁻³ This approach allows for a more focused, efficient, and comprehensive pathologic analysis of micrometastatic disease. IHC analysis using S-100-, HMB-45-, and MART-1-specific antibodies (Abs) has demonstrated a 10% to 30% improved sensitivity for identifying micrometastases over conventional hematoxylin and eosin (H&E) staining.⁴⁻⁷ Additional upstaging of patients who were shown to have significantly poorer prognoses by a multivariate analysis has been obtained utilizing a multimarker quantitative real-time reverse-transcription PCR (qRT) for diagnosing melanoma metastasis in SLN.⁸ Nevertheless, up to 20% of patients, depending on institute, with tumor-negative SLNs will develop recurrent disease.^{8,9} This suggests that occult micrometastasis may be missed by IHC.

HMW-MAA, also known as the melanoma chondroitin sulfate proteoglycan, is expressed in >85% of primary and metastatic melanoma lesions with limited inter- and intra-lesional heterogeneity.¹⁰ MART-1-specific Ab has been shown to effectively detect melanomas by IHC, and studies have shown that it is equivalent or more sensitive and specific than S-100- and HMB-45-specific Abs for the evaluation of SLN micrometastases.^{11,12} The sensitivity and specificity of IHC biomarkers in detecting melanoma metastasis needs improvement. The use of multiple types of antibodies for tissue assessment is logistically cumbersome and requires more tissue sections to be assessed. In the present study, whether the sensitivity and accuracy of diagnosis of SLN melanoma metastasis could be enhanced by using HMW-MAA cocktail mAbs as an IHC biomarker has been determined. Moreover, IHC analysis using HMW-MAA mAb with the standard IHC analysis for SLN of melanoma using MART-1-specific mAb was compared.

### Materials and Methods

### Cell Lines

The human metastatic melanoma cell lines, ME-01, ME-02, ME-05, ME-09, ME-10, ME-13, ME-16, ME-17, ME-18, ME-19, ME-20, ME-35, and ME-36 were grown at 37° C. in a 5% CO2 humidified atmosphere in RPMI 1640 (Gibco-BRL Life Technologies, Gaithersburg, Md.) medium supplemented with 10% fetal bovine serum. Peripheral blood lymphocytes (normal PBL) were harvested from normal consenting healthy donors, G595, G596, G597, G598, G599, G600, G601, G602, G603, and PBL-CP298.

### Patients

Informed human subject consent, approved by Saint John's Health Center (SJHC, Santa Monica, Calif.)/John Wayne Cancer Institute (JWCI) institutional review board, was obtained for all patient specimens. All surgical LN tissues used from 1995 to 2006 were obtained in consultation with surgeons and pathologists at JWCI. Eligible patients who received surgery for SLN or LN dissection of melanoma between 1995 and 2006 were initially identified and then sequentially selected based on available PEAT SLN or LN blocks. All surgery SLN patients were diagnosed with early-stage clinically SLN-negative malignant melanoma and underwent preoperative lymphoscintigraphy to identify the tumor-draining LN basin(s). SLN dissection was performed after intraoperative lymphatic mapping of the SLNs with a combination of isosulfan blue dye (Lymphazurin; Hirsch Industries Inc., Richmond, Va.) and a radioisotope (99m technetium sulfur colloid).¹⁻³ Fifty-eight melanoma patients were selected based on the above defined criteria by the melanoma database management personnel, independently of investigators and biostatisticians.

All SLN (n=58) tissues were stained with H&E, and most were stained by IHC using S-100-, HMB-45-, and MART-1-specific Abs in the Department of Pathology at SJHC (RRT).^{2,3} The slides were reviewed by a surgical pathologist, and 42 SLN tissues were diagnosed as melanoma-positive. The size of the metastatic melanoma deposit in each SLN was assessed as previously described, and defined as a macrometastasis (>2 mm) (n=10) or micrometastasis (>=2 mm) (n=32).^{13,14} Fifty-two LN macrometastasis (10 SLN macrometastasis and 42 melanoma-positive LN tissues) and 32 SLN micrometastasis tissues were assessed in this study. Sixteen melanoma-negative SLN tissues were used as normal LNs for negative control tissues.

### Monoclonal Antibodies

The mAb 763.74, VF1-TP41.2, and VT80.12, which recognize distinct determinants of HMW-MAA, were developed and characterized as described. mAb were purified from ascitic fluid by sequential precipitation with caprylic acid and ammonium sulfate. The purity of mAb preparations was assessed by SDS-PAGE; activity was assessed by ELISA with HMW-MAA-positive melanoma cells. A cocktail of the three mAbs, each at a final concentration of 0.5 mg/ml, was used as a probe in immunohistochemical assays. MART-1-specific mAb (M2-7C10) and a secondary anti-mouse immunoglogulin-HRP were purchased from GeneTex, Inc, San Antonio, Tex. and DakoCytomation, Carpinteria, Calif., respectively.

### Immunohistochemistry

Immunohistochemical staining was performed on PEAT (5 μm sections). Tissues were sectioned, incubated overnight at 50° C., and deparaffinized in xylene. CSA II, Biotin-Free Catalyzed Amplification System (DakoCytomation) was modified using HMW-MAA mAb as follows. Tissue sections were treated for Antigen Retrieval: 1 mM EDTA, pH 8.0, heated to the boiling point for 15 min, and then cooled to room temperature for 20 min. After three rounds of TBST washing for 5 min each, endogenous peroxidase was quenched with Peroxidase Block (CSA II) for 5 min at room temperature. Nonspecific binding was blocked by a 5 min incubation at room temperature with Protein Block Serum-Free (CSA II). Tissue sections were then incubated overnight at 4° C. with the HMW-MAA-specific mAb pool at a final concentration of 15 ug/ml. Negative controls were incubated with normal mouse IgG (Santa Cruz Biotechnology, Santa Cruz, Calif.) under the same experimental conditions. Following washings, tissue sections were incubated for 15 min at room temperature with a secondary Anti-Mouse Immunoglogulin-HRP (CSA II). Following amplification with Amplification Reagent (CSA II) for 15 min at room temperature, anti-Fluorescein-HRP (CSA II) was applied and incubation was continued for an additional 15 min at room temperature. After development with the Vector VIP Kit, tissue sections were counterstained with 1× Gill Hematoxylin (Fisher Scientific Company, Middletown, Va.) for 1 min at room temperature, dehydrated, and mounted.

Standard procedures were utilized for immunohistochemical staining of tissue sections with MART-1-specific mAb M2-7C10. After deparaffinization, endogenous peroxidase was quenched with Peroxidase Block (Fisher Scientific). Sections were then incubated via Antigen Retrieval 10 mM Citrate Buffer, pH 6.0 (DBS, Pleasanton, Calif.) at 120° C. for 20 min, then cooled to room temperature for 20 min in phosphate buffered saline (PBS) (Invitrogen Corporation, Carlsbad, Calif.). Protein Block (DakoCytomation) was used for blocking protein. Sections were incubated with mAb M2-7C10 at room temperature for 60 min. After three rounds of PBS washing at 5 min each, sections were incubated with EnVision+System Labelled Polymer-HRP Anti-Mouse Ab (DakoCytomation) for 30 min, then three more rounds of PBS washing at 5 min each. AEC Substrate Chromogen (DakoCytomation) was used for the development process for 10 min, followed by 5 min of PBS washing. Sections were counterstained via Gill Hematoxylin (Fisher Scientific, Pittsburgh, Pa.) for 1 min, and then mounted.

### Scoring of Tissue Sections

Tissue sections were scored according to the percentage of stained melanoma cells as 100-75%, 75-50%, 50-25%, >25%, and negative. The intensity of staining was scored as strong, intermediate, weak, and negative. All tissue sections were reviewed by three independent observers. The staining of each tissue section was scored as the average percentage of stained cells and was assessed by three independent observers.

### RNA Isolation

Total RNA was extracted from melanoma cells, normal PBL, and PEAT LN blocks using the Tri-Reagent (molecular Research Center, Inc., Cincinnati, Ohio), as previously described.^{15,16} LN metastasis tissues of melanoma were selected from the same PEAT blocks as those used for IHC. LN macrometastases (n=31), SLN micrometastases (n=17), and normal LNs (n=10) were selected based on the availability of PEATs for both the qRT and IHC assays. Five 10 μm-thick sections were cut from each LN PEAT block with a sterile microtome blade and placed in sterile microcentrifuge tubes (Eppendorf, Westbury, N.Y.).⁸ After deparaffinization, specimens were treated with a proteinase K digestion buffer for 3 hr before RNA extraction, as previously described.¹⁸ Total RNA was extracted, isolated, and purified using a modified RNAWiz (Ambion, Austin, Tex.) phenol-chloroform extraction method, as previously described.^{17,18} RNA was quantified and assessed for purity by ultraviolet spectrophotometry and a RIBOGreen detection assay, as previously described (Molecular Probes, Eugene, Oreg.).¹⁹

### Primers and RT-PCR

Primer and probe sequences were designed for the qRT assay, as previously described.²⁰ Fluorescence resonance energy transfer (FRET) probe sequences were designed to enhance the specificity of the assay. Specific primers were designed to sequence at least one exon-exon region. The HMW-MAA primer sequence was: 5′-TGGAAGAACAAAGGTCTCTGG-3′ (forward); 5′-GCTGGCCAAGAGATTGGAG-3′ (reverse). The HMW-MAA (FRET) probe sequence was: 5′-FAM-AGGATCACCGTGGCTGCTCT-BHQ-1-3′. The MART-1 primer sequence was: 5′-AAAACTGTGAACCTGTGGT-3′ (forward); 5′-TTCAAGCAAAAGTGTGAGAGA-3′ (reverse). The MART-1 FRET probe sequence was: 5′-FAM-CAGAACAGTCACCACCACCTTATT-BHQ-1-3′. The glyceraldehyde-3-phosphate dehydrogenase (GAPDH) primer sequence was: 5′-GGGTGTGAACCATGAGAAGT-3′ (forward); 5′-GACTGTGGTCATGAGTCCT-3′ (reverse). The GAPDH FRET probe sequence was: 5′-FAM-CAGCAATGCCTCCTGCACCACCAA-BHQ-1-3′. Expression of housekeeping gene GAPDH served as an internal reference for mRNA integrity.

qrT

The qRT assay was performed on the iCycler iQ RealTime PCR Detection System (Bio-Rad Laboratories, Hercules, Calif.) using 250 ng total RNA per reaction. The PCR mixture consisted of 0.4 μM of each primer, 0.3-μM TaqMan probe, 1 unit of AmpliTaq Gold polymerase (Applied Biosystems, Foster City, Calif.), 200 μM each of deoxynucleotide triphosphate, 4.5 mM MgCl₂, and AmpliTaq buffer diluted to a final volume of 25 μL. Samples were amplified with a pre-cycling hold at 95° C. for 10 min, followed by 35 cycles of denaturation at 95° C. for 1 min, annealing for 1 min at 55° C. for GAPDH, 63° C. for HMW-MAA, and 59° C. for MART-1, and extension at 72° C. for 1 min. Absolute copy numbers were determined by a standard curve with serial dilutions (10⁶-10¹ copies) of HMW-MAA, MART-1, and GAPDH cDNA templates. PCR efficiency evaluated from the slopes of the curves was between 95% and 100%. The correlation coefficient for all standard curves was 0.99. The product size of HMW-MAA, MART-1, and GAPDH was confirmed by gel electrophoresis, and then the assay conditions for qRT were optimized, as previously described.^{13,14} HMW-MAA mRNA expression was designated as relative mRNA copies (absolute mRNA copies of HMW-MAA/absolute mRNA copies of GAPDH) to compensate for comparison of different assays. Each sample was assayed in triplicate with positive and reagent negative controls.

### Statistical Analysis

The Wilcoxon signed rank test was used to analyze the difference in percentage and intensity of staining between MART-1 and HMW-MAA. The Wilcoxon rank sum test was used to assess the difference in HMW-MAA and MART-1 mRNA expression between melanoma cell lines and normal PBL, and between LN macrometastases, SLN micrometastases, and normal LN tissues. The Fisher's exact test was used to assess the frequency of HMW-MAA and MART-1 expression in LN metastasis tissues by IHC and qRT. Analysis was performed using SAS statistical software (SAS Institute, Cary, N.C.), and all tests were two-sided with a significance level of P<0.05.

### Results

### HMW-MAA IHC

Before IHC using HMW-MAA mAb on LN metastases of melanoma patients was investigated, the presence of HMW-MAA protein on melanoma cell surface was assessed using an HMW-MAA mAb cocktail. Using PEAT primary and various organs metastatic melanomas, IHC using HMW-MAA mAb was optimized, and HMW-MAA was clearly observed in the membrane of melanoma cells.

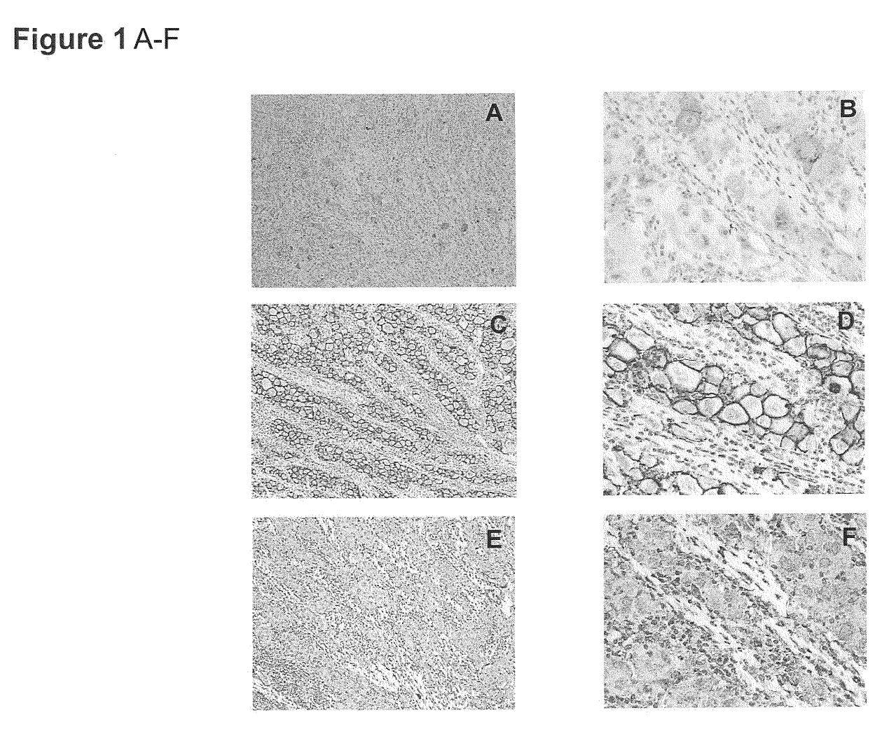

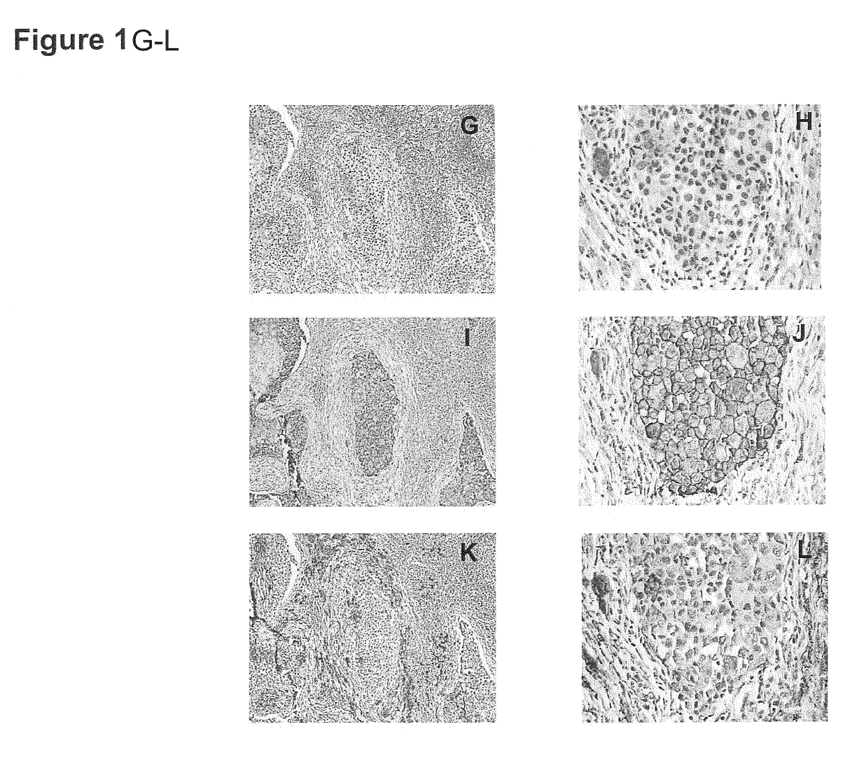

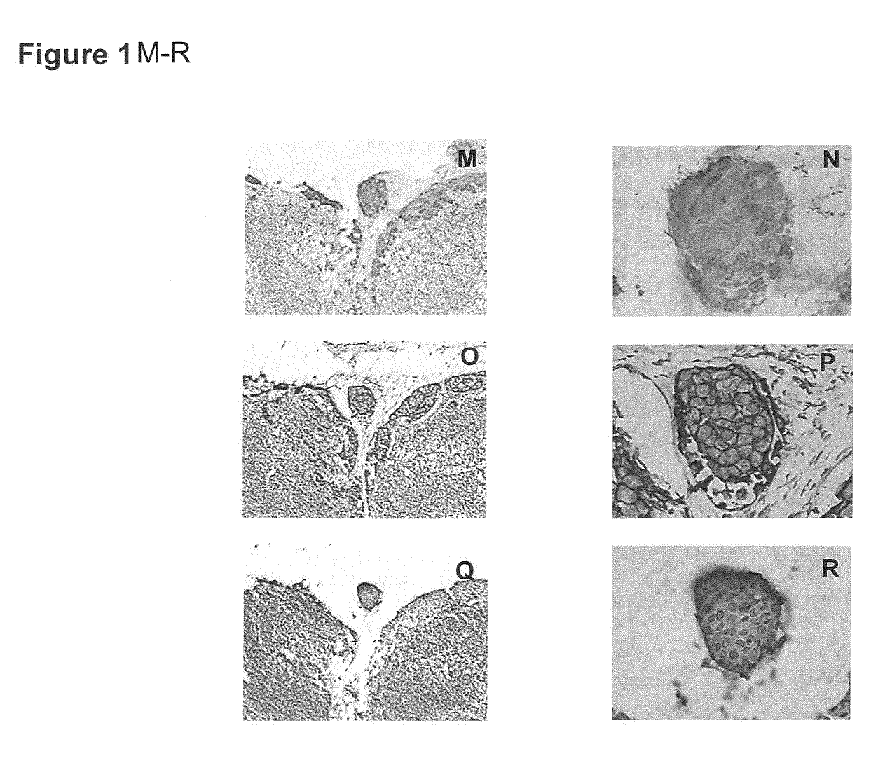

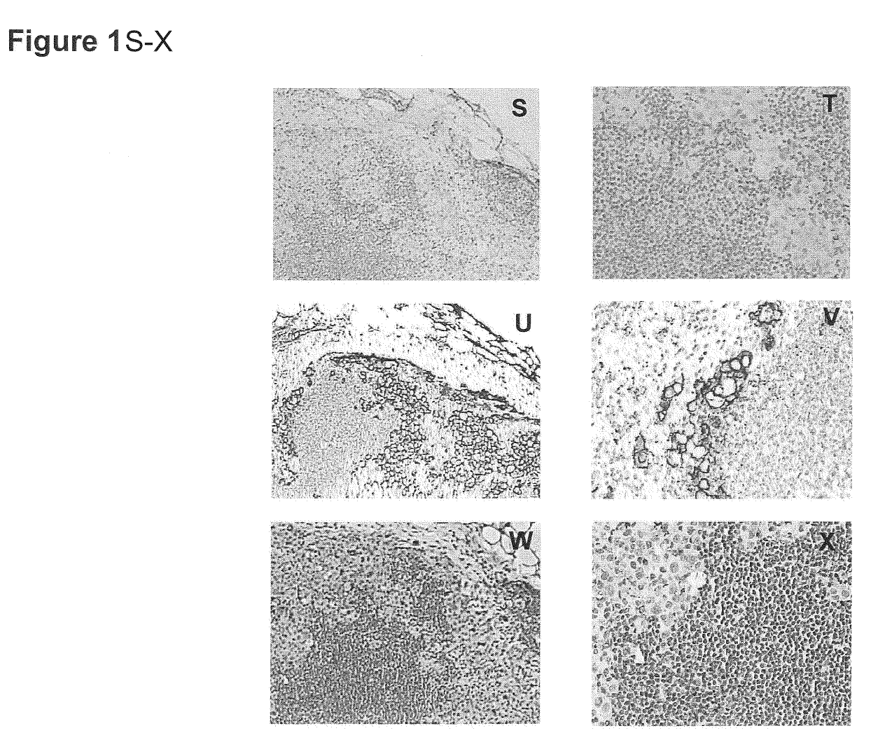

HMW-MAA mAb was used to investigate LN macrometastases, including SLN macrometastases of melanomas (n=52), SLN micrometastases of melanomas (n=32), and normal LN (n=16). IHC of LN macrometastases resulted in membrane staining of melanoma cells by HMW-MAA-specific mAb cocktail (FIG. 1). The staining is specific, since melanoma cells were not stained by normal mouse IgG. Furthermore, lymphocytes surrounding melanoma cells were not stained by HMW-MAA-specific mAb pool (FIG. 1). The staining patterns of all LN metastases of melanomas in terms of percentage of stained melanoma cells and staining intensity are shown in Table 1. Melanoma cells were stained by HMW-MAA-specific Ab in all LN metastases, including SLN micrometastases, but not in normal LN tissues.

TABLE 1
Distribution of IHC Intensity and Frequency of
LN Metastasis (n = 84) by HMW-MAA-specific mAb

| | Percentage of Melanoma | | | | | |
|---|---|---|---|---|---|---|
| Intensity of Staining in | Cells (+) in a Lesion | | | | | |
| Positive Melanoma Cells* | 100% | <75% | 50-75% | 25-50% | >25% | 0% |
| +++ | 16 | 0 | 0 | 0 | 0 | 0 |
| ++ | 14 | 21 | 9 | 1 | 2 | 0 |
| + | 2 | 8 | 6 | 3 | 2 | 0 |
| − | 0 | 0 | 0 | 0 | 0 | 0 |

*Intensity of staining in positive melanoma cells, +++: Strong. ++: Intermediate, +: Weak, −: Negative.

Comparison of HMW-MAA mAb IHC with S-100 and HMB-45 Ab IHC

After SLND, most SLNs were stained with IHC using S-100-specific Ab (rabbit polyclonal Ab) and HMB-45-specific mAb in the Department of Pathology at Saint John's Medical Center (RRT). HMW-MAA-specific mAb in IHC was compared to S-100- and HMB-45-specific Abs. All 7 SLN macrometastasis tissues were stained by S-100-, HMB-45-, and HMW-MAA Abs (Table 2A). In SLN micrometastases, whereas 21 of 23 (91%) and 18 of 23 (78%) tissues were stained by S-100 and HMB-45 Abs, respectively, all 23 tissues were stained by HMW-MAA-specific mAb (Table 2B). These findings indicate that for detecting SLN micrometastases, HMW-MAA mAb is equivalently or more sensitive than S-100, and more sensitive than HMB-45-mAb, whereas HMW-MAA, MART-1, and HMB-45 Abs are sensitive for detecting SLN macrometastases.

TABLE 2A
Comparison of HMW-MAA mAb IHC with S-100 and HMB-45
Ab IHC in SLN Macrometastases

| | Melanoma | | |
|---|---|---|---|
| Patient | S100 | HMB45 | HMW-MAA |
| 1 | + | + | + |
| 2 | + | + | + |
| 3 | + | + | + |
| 4 | + | + | + |
| 5 | + | +/− | + |
| 6 | + | + | + |
| 7 | + | + | + |
| Total | 7/7 (100%) | 7/7 (100%) | 7/7 (100%) |

TABLE 2B
Comparison of HMW-MAA mAb IHC with S-100 and HMB-45
Ab IHC in SLN micrometastases

| | Melanoma | | |
|---|---|---|---|
| Patient | S100 | HMB45 | HMW-MAA |
| 1 | − | +/− | + |
| 2 | + | + | + |
| 3 | − | + | + |
| 4 | + | − | + |
| 5 | + | + | + |
| 6 | + | − | + |
| 7 | + | − | + |
| 8 | + | +/− | + |
| 9 | + | + | + |
| 10 | + | + | + |
| 11 | + | − | + |
| 12 | + | + | + |
| 13 | + | + | + |
| 14 | + | + | + |
| 15 | + | +/− | + |
| 16 | + | +/− | + |
| 17 | + | + | + |
| 18 | + | +/− | + |
| 19 | + | + | + |
| 20 | + | + | + |
| 21 | + | + | + |
| 22 | + | + | + |
| 23 | + | − | + |
| Total | 21/23 (92%) | 18/23 (78%) | 23/23 (100%) |

Comparison of HMW-MAA IHC with MART-1 IHC

IHC analysis using HMW-MAA Ab was compared with the standard IHC analysis for SLN of melanoma using MART-1-specific mAb in optimal conditions. MART-1 Ab was used to investigate the same PEAT LNs as those for HMW-MAA Ab. In the melanoma cells, MART-1 protein expression was observed in the cytoplasm (FIG. 1). The result of all LN metastases was divided into two groups, LN macrometastases, including SLN macrometastases, and SLN micrometastases of melanomas. In both LN macrometastases and SLN micrometastases, the intensity of staining by HMW-MAA Ab was stronger than that of MART-1 Ab (Table 3A, P<0.0001 and Table 3C, P=0.004). Whereas 43 of 52 (83%) LN macrometastases were found to have MART-1, all of 52 (100%) specimens had HMW-MAA (Table 3A). All 33 (100%) SLN micrometastases demonstrated HMW-MAA staining; only 22 of 32 (69%) had MART-1 (Table 3C). The frequency (percentage of stained melanoma cells in a lesion) of HMW-MAA was higher than that of MART-1 in both LN macrometastases and SLN micrometastases (Table 3B, P<0.0001 and Table 3D, P<0.0001). A majority (>50%) of melanoma cells were stained using MART-1-specific mAb in 28 of 52 (53%) LN macrometastases of melanomas, while 43 of 53 (90%) were stained by IHC using HMW-MAA-specific mAb (Table 3B, P<0.0001). A majority (>50%) of melanoma cells were stained by IHC using MART-1-specific mAb in 16 of 32 (50%) SLN micrometastases of melanomas; a majority of cells in 29 of 32 SLN micrometastases (91%) were stained by IHC using HMW-A-specific mAb (Table 3D, P=0.0023). These results indicate that IHC using HMW-MAA-specific mAb is more sensitive and stains more intensely than IHC using MART-1-specific mAb when used to detect LN metastases and SLN macro- and micrometastases of melanoma. In addition, anti-HMW-MAA Ab can detect occult tumor cells that are not detected by anti-MART-1 Ab.

TABLE 3A
Comparison of IHC Intensity of LN Macrometastasis (n = 52)
by HMW-MAA and MART-1 mAbs

| | |
|---|---|
| HMW-MAA (%) | MART-1 (%) |

| Staining Intensity | Specimens | Staining Intensity | Specimens |
|---|---|---|---|
| +++ | 11 (21) | +++ | 1 (2) |
| ++ | 28 (54) | ++ | 20 (38) |
| + | 13 (25) | + | 22 (42) |
| − | 0 (0) | − | 9 (17)* |

*+++; Strong. ++: Intermediate, +: Weak, −: Negative
*P < 0.0001

TABLE 3B
Comparison of IHC Frequency of LN Macrometastasis (n = 52)
by HMW-MAA and MART-1 mAbs

| HMW-MAA (%) | | MART-1 (%) | |
|---|---|---|---|
| Frequency of Positive | | Frequency of Positive | |
| Melanoma Cells | Specimens | Melanoma Cells | Specimens |
| 100% | 23 (44) | 100% | 12 (23) |
| 75-100% | 17 (33) | <75% | 7 (13) |
| 50-75% | 7 (13) | 50-75% | 9 (17) |
| 25-50% | 2 (4) | 25-50% | 8 (15) |
| >25% | 3 (6) | >25% | 7 (13) |
| — | 0 (0) | — | 9 (17)* |
| >50% | 47 (90) | >50% | 28 (53) |

*P < 0.0001

TABLE 3C
Comparison of IHC Intensity of SLN Micrometastasis (n = 32)
by HMW-MAA and MART-1 mAbs

| | |
|---|---|
| HMW-MAA (%) | MART-1 (%) |

| Staining Intensity | Specimens | Staining Intensity | Specimens |
|---|---|---|---|
| +++ | 5 (16) | +++ | 3 (9) |
| ++ | 19 (59) | ++ | 13 (41) |
| + | 8 (25) | + | 6 (19) |
| − | 0 (0) | − | 10 (31)* |

TABLE 3D
Comparison of IHC Frequency of SLN Micrometastasis (n = 32)
by HMW-MAA and MART-1 mAbs

| HMW-MAA (%) | | MART-1 (%) | |
|---|---|---|---|
| Frequency of Positive | | Frequency of Positive | |
| Melanoma Cells | Specimens | Melanoma Cells | Specimens |
| 100% | 9 (28) | 100% | 3 (9) |
| <75% | 12 (38) | <75% | 7 (22) |
| 50-75% | 8 (25) | 50-75% | 6 (19) |
| 25-50% | 2 (6) | 25-50% | 4 (13) |
| >25% | 1 (3) | >25% | 2 (6) |
| — | 0 (0) | — | 10 (31)* |
| >50% | 29 (91) | >50% | 16 (50) |

*P < 0.0001

Detection of HMW-MAA mRNA in LN Metastases

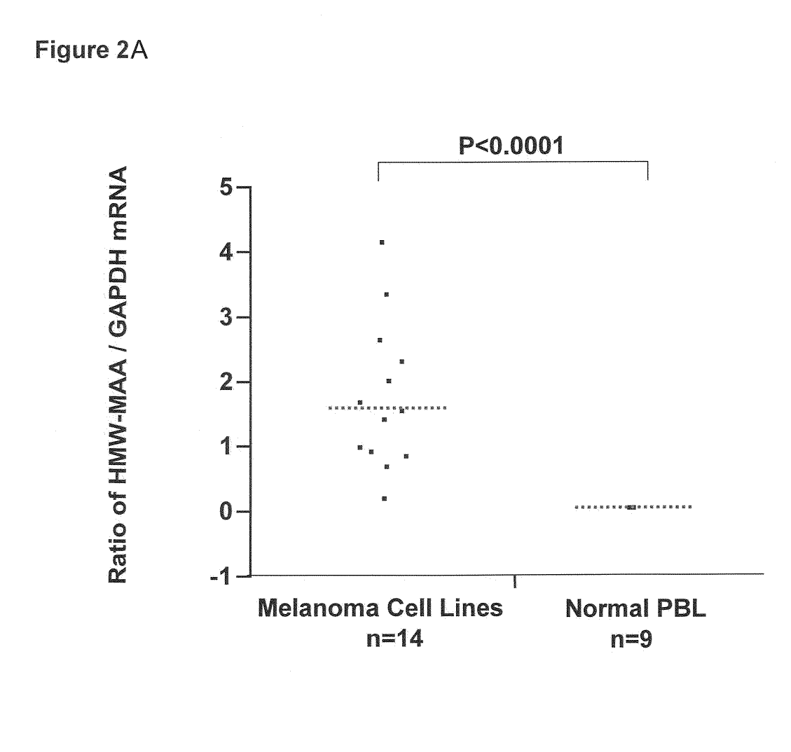

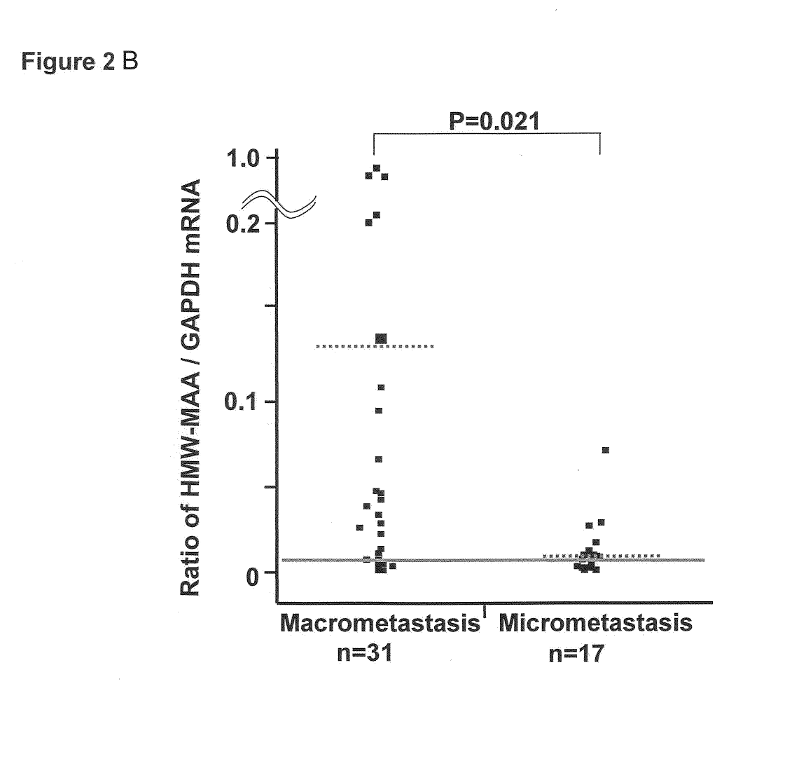

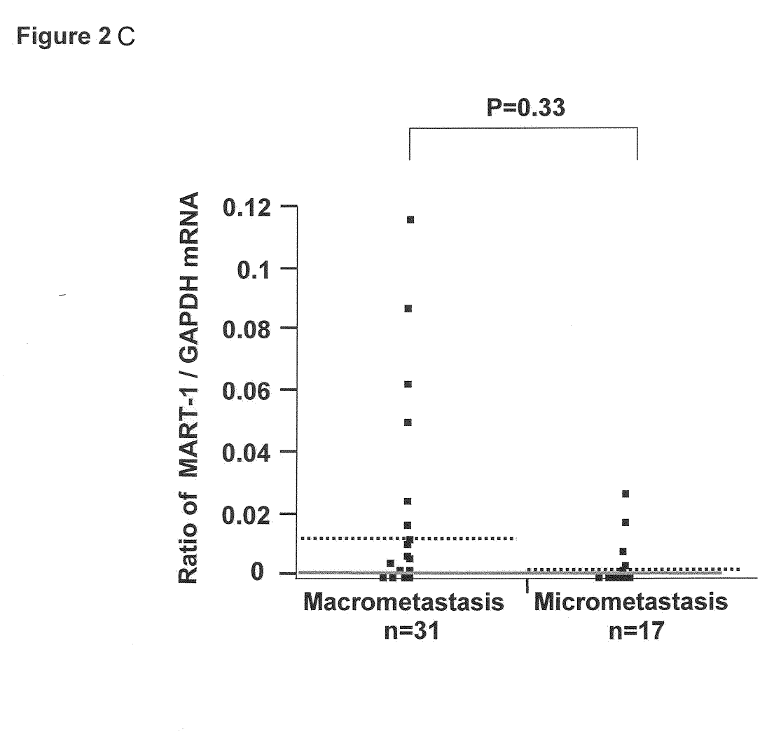

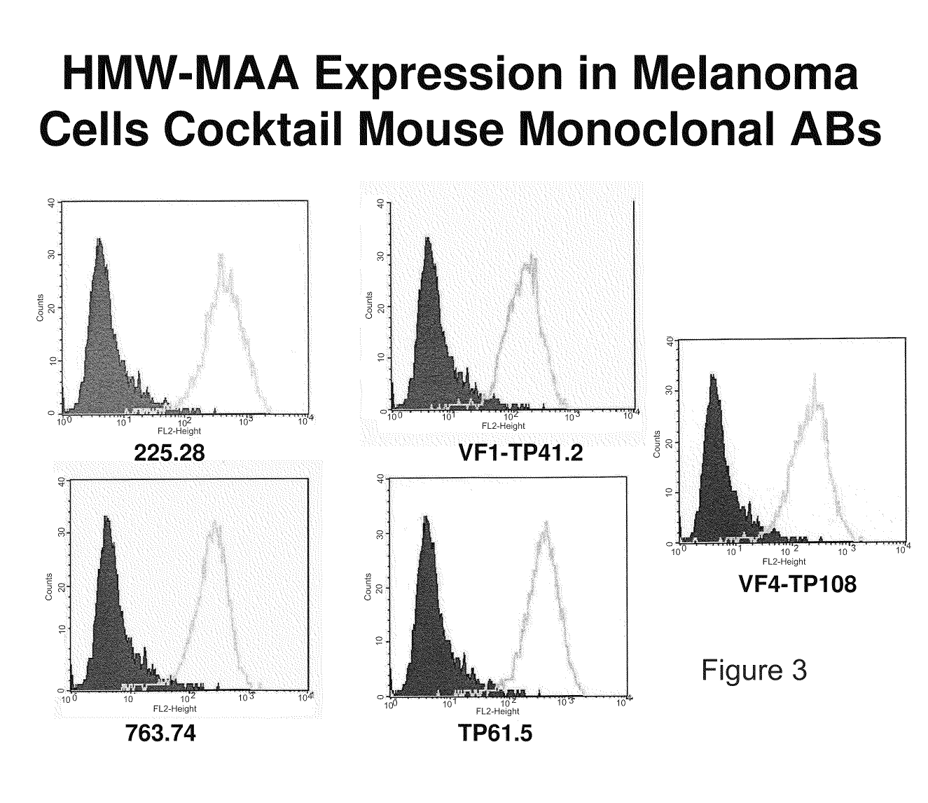

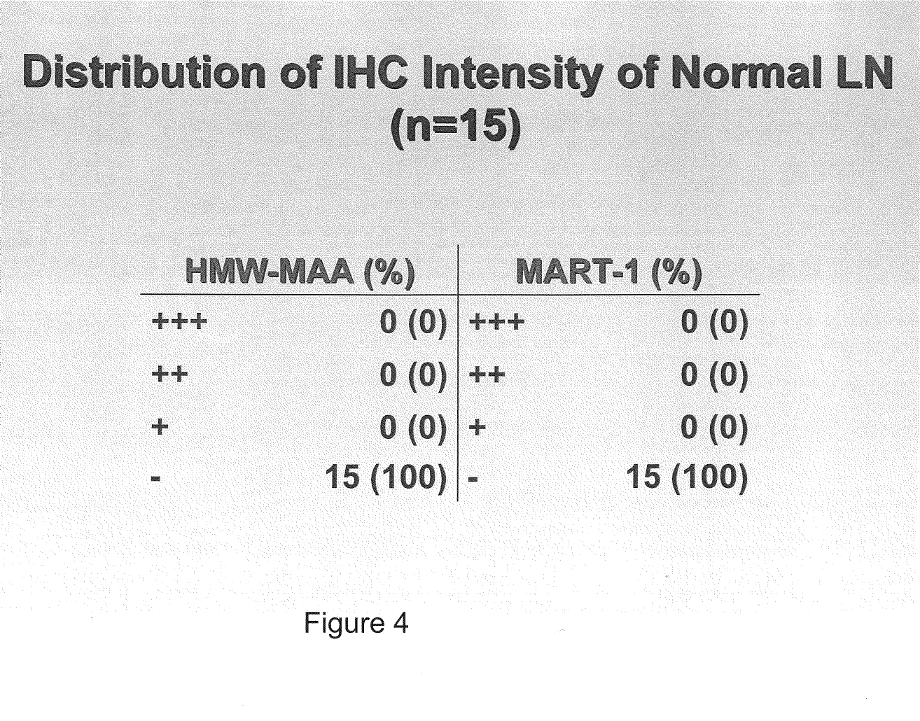

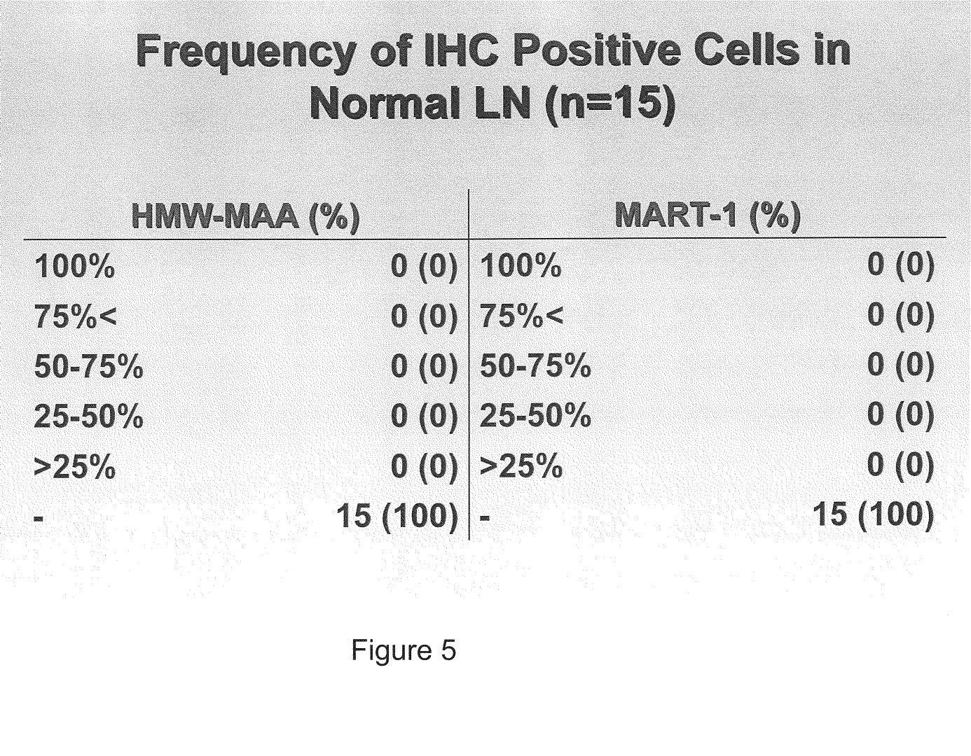

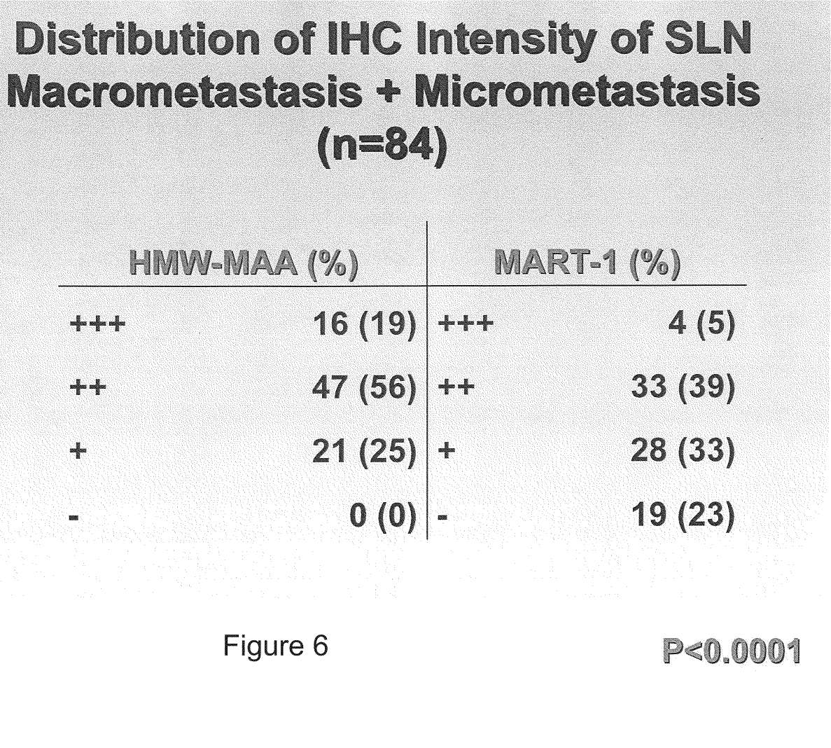

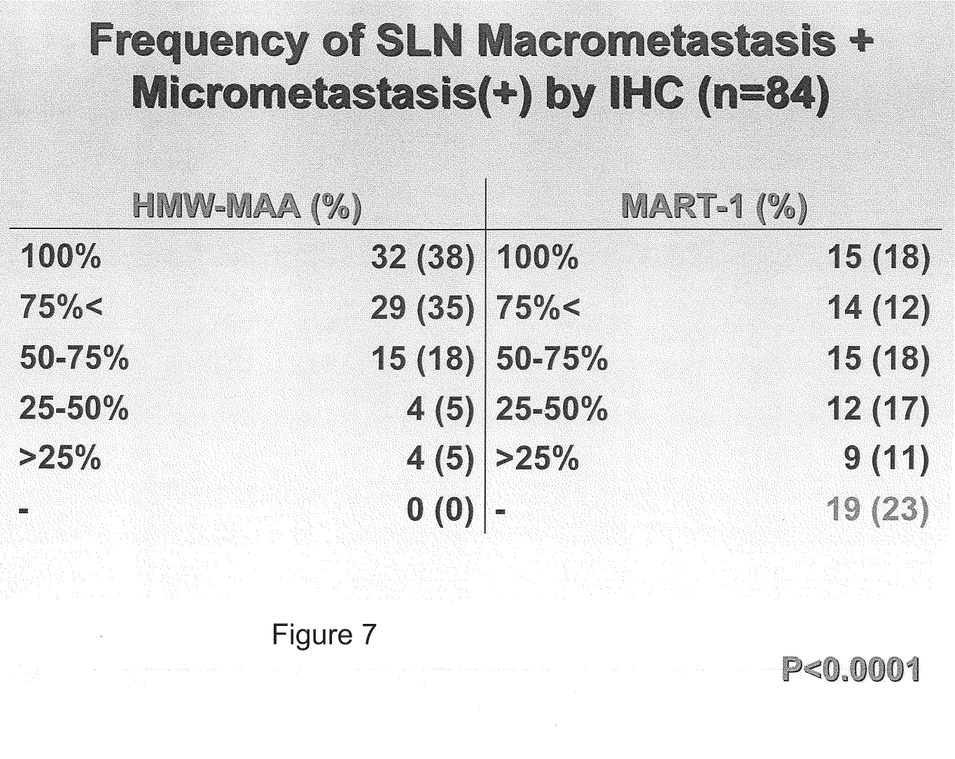

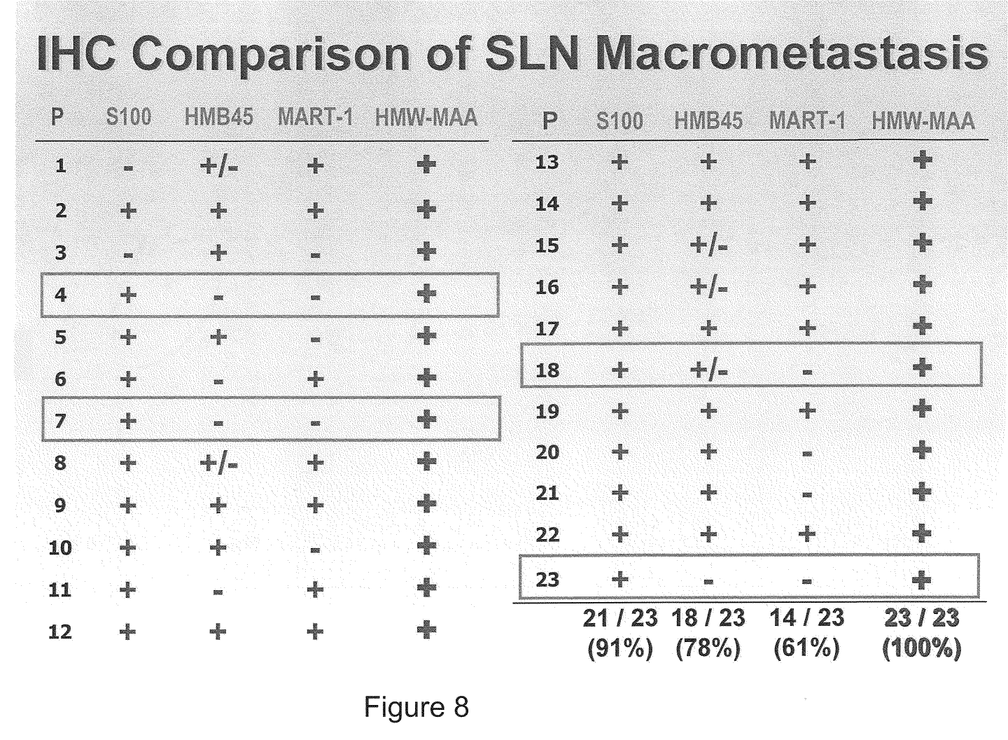

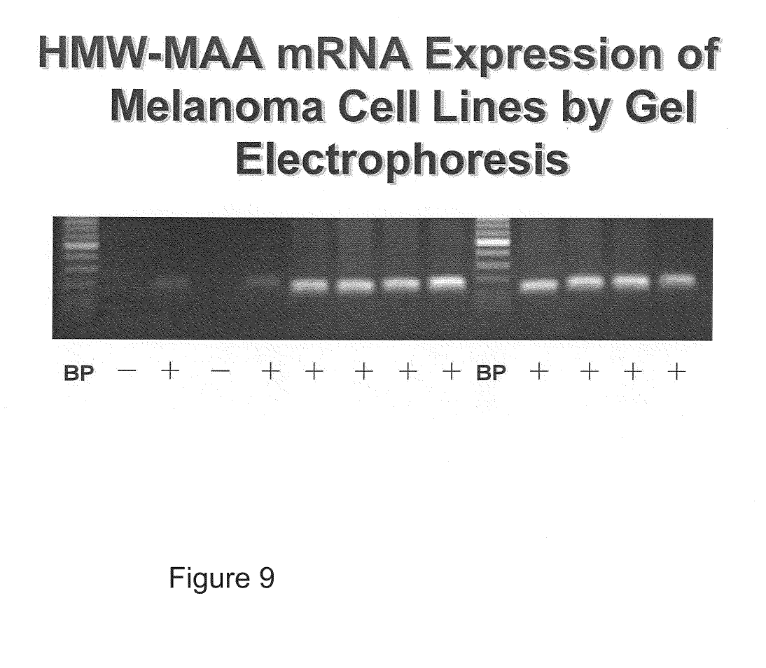

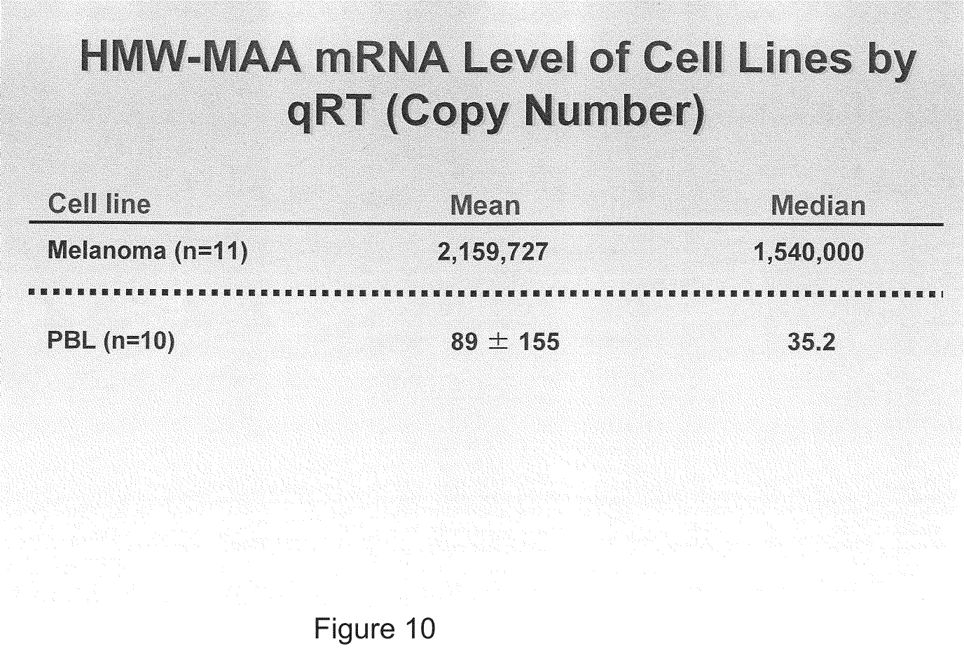

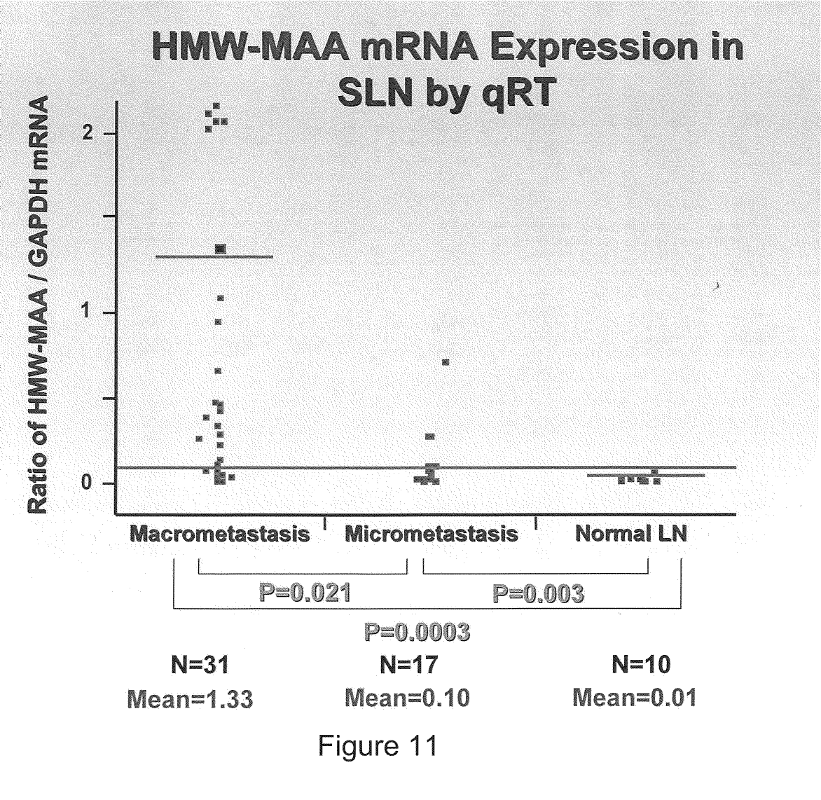

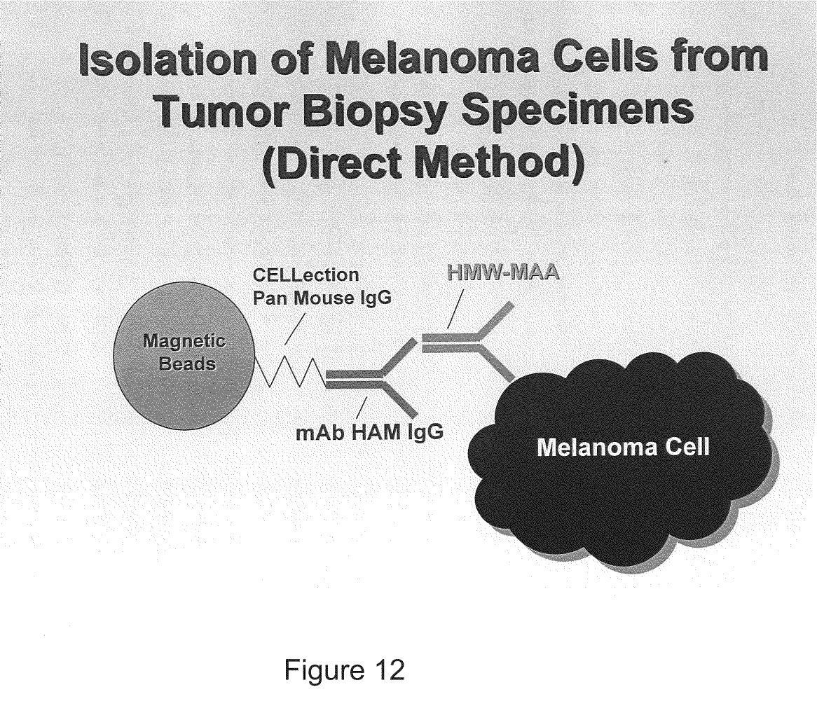

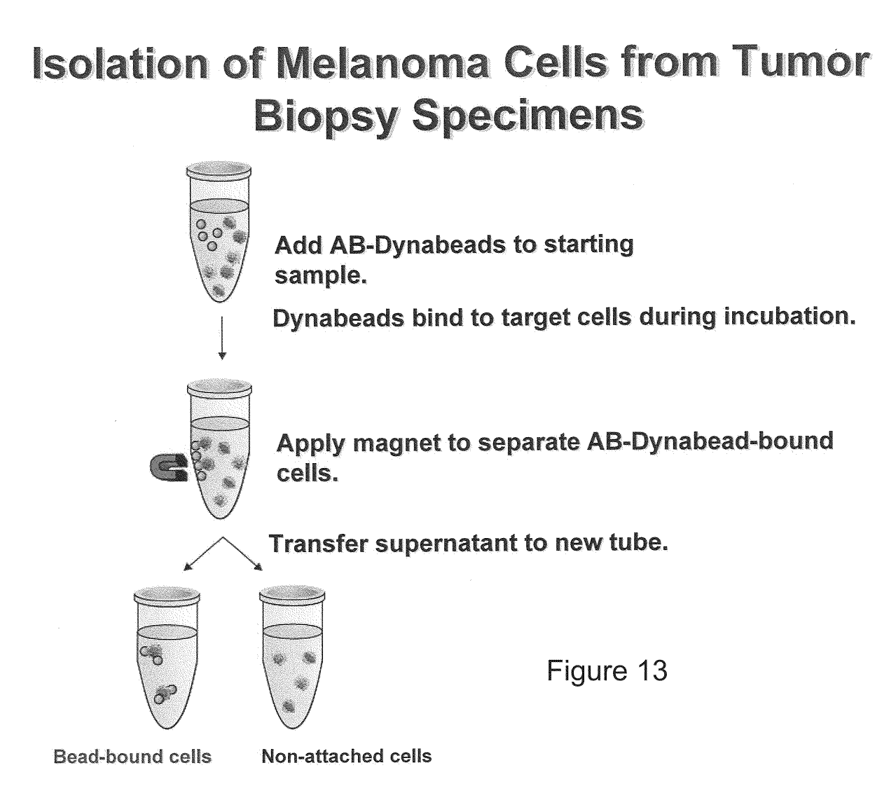

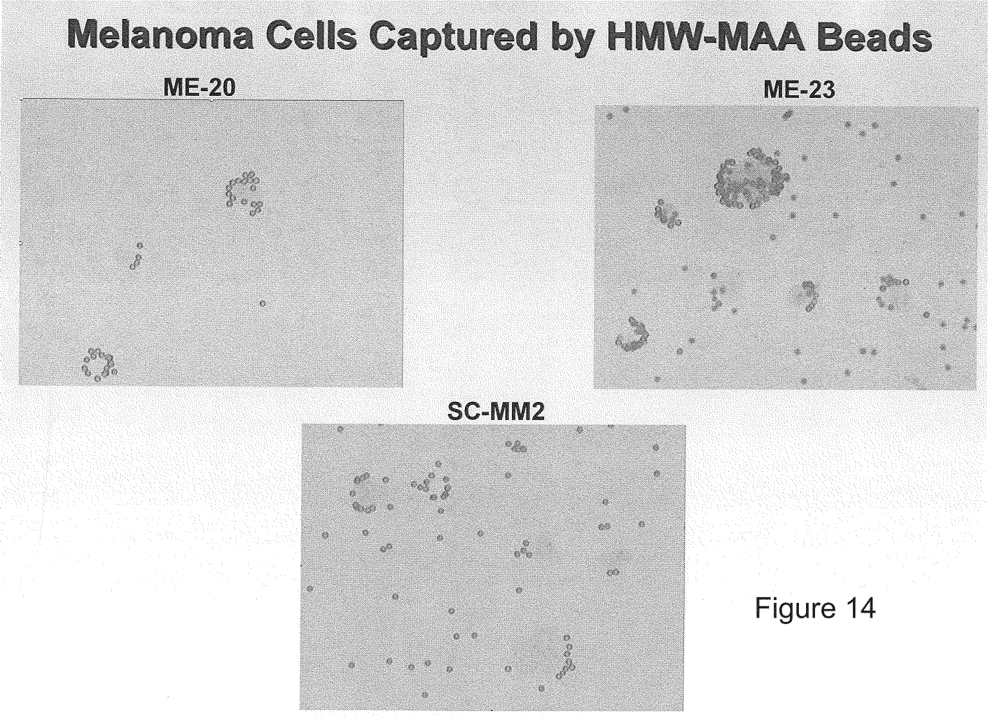

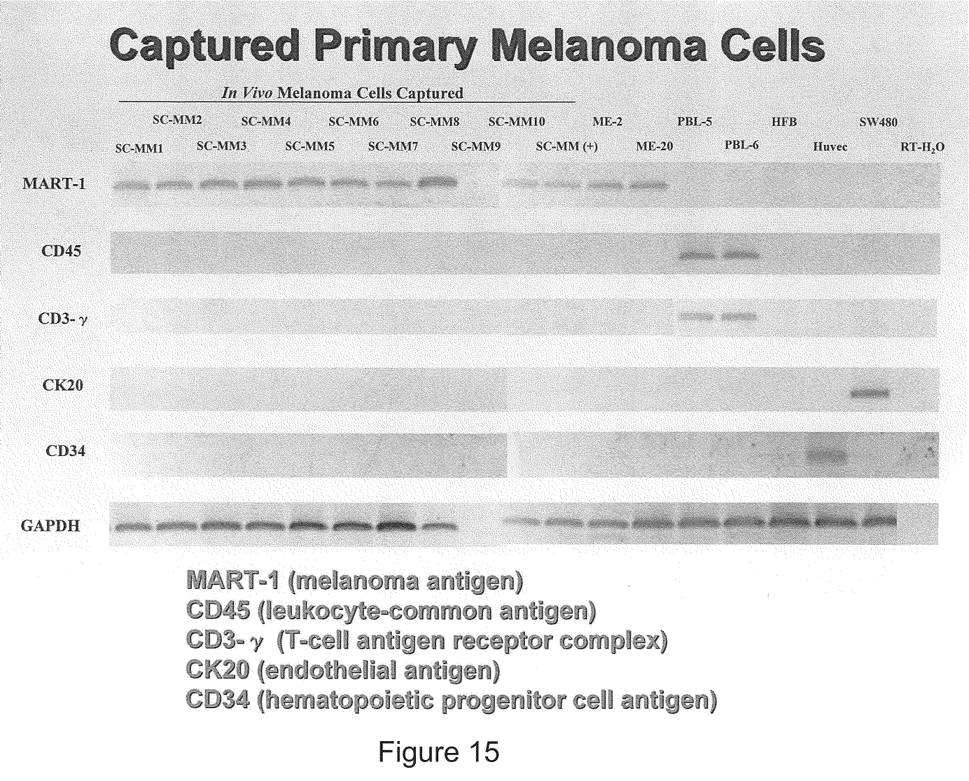

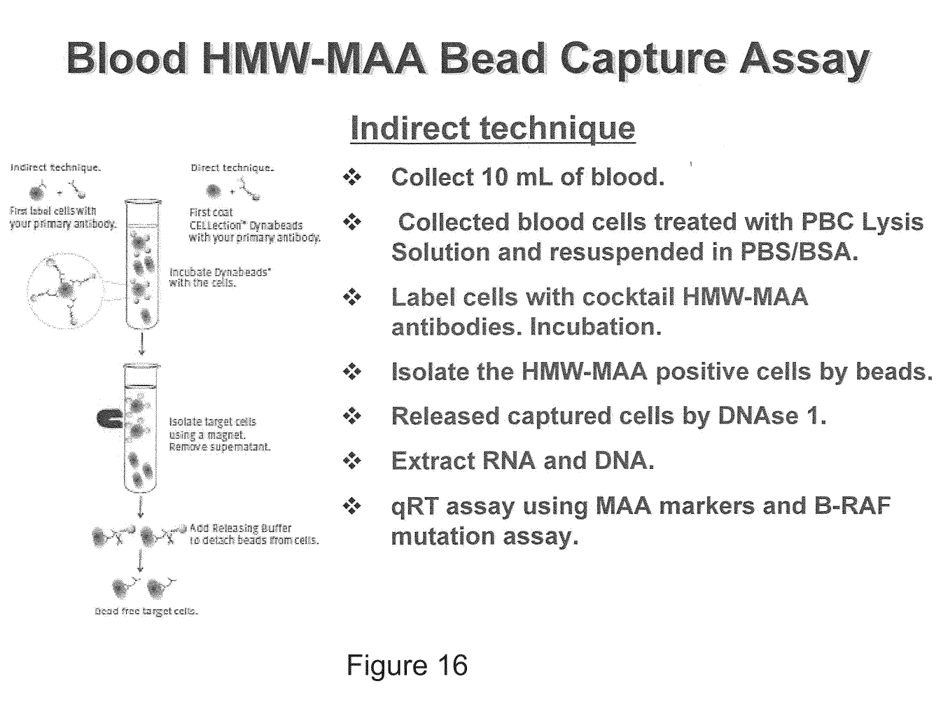

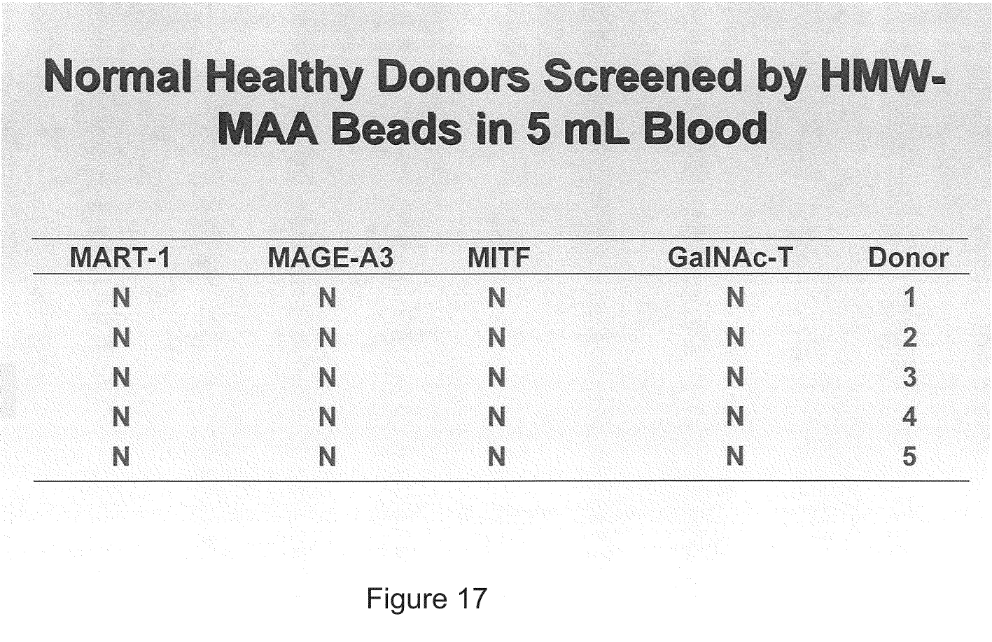

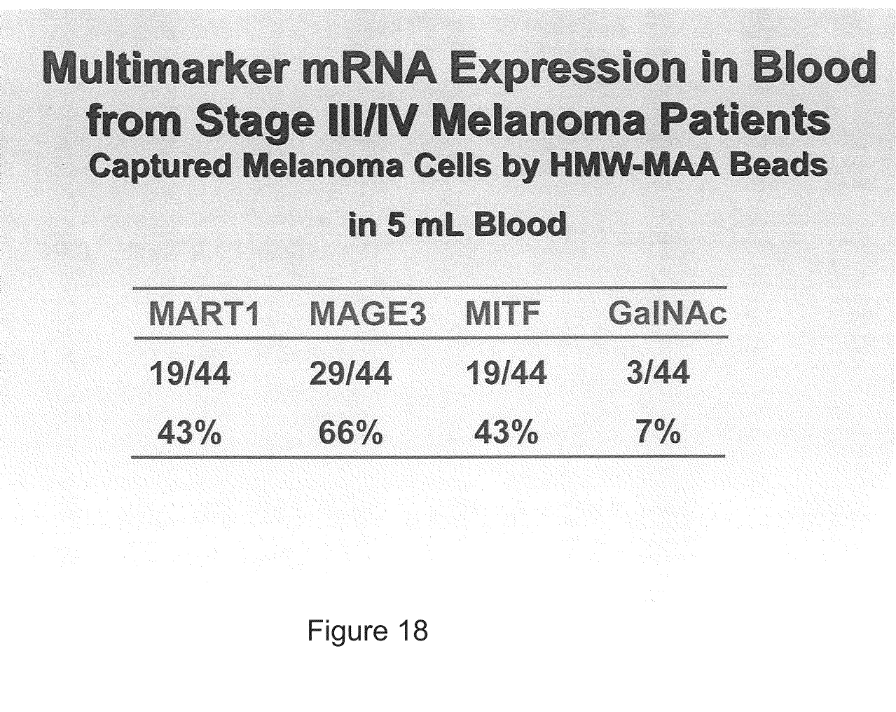

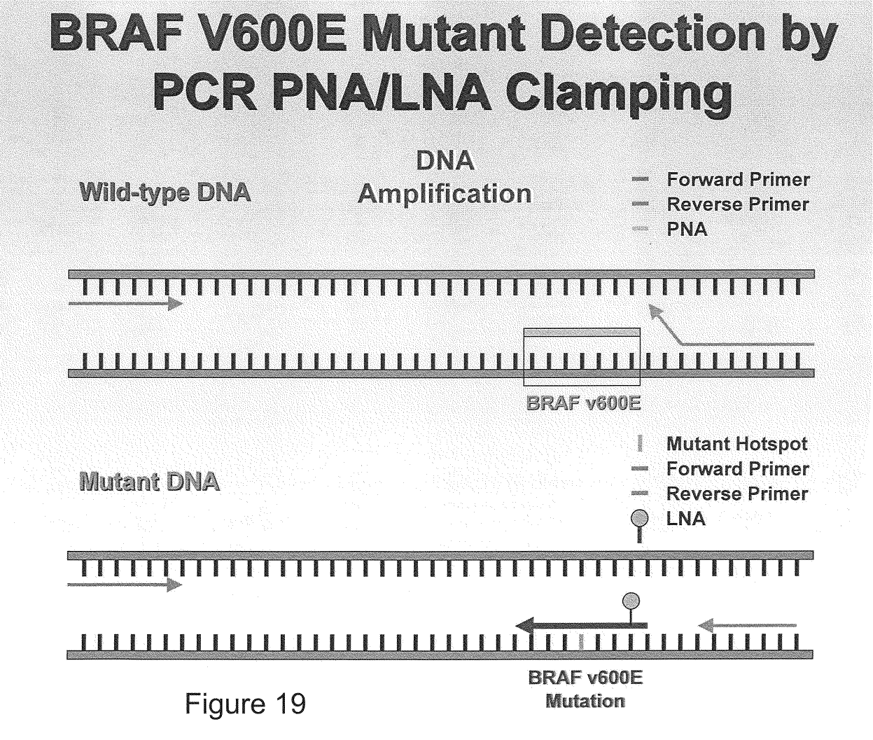

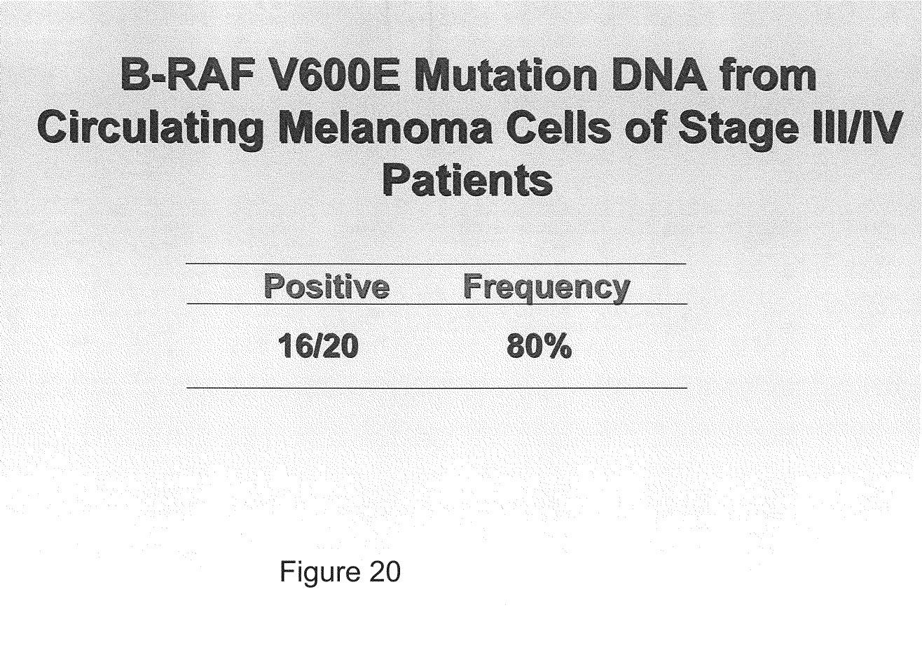

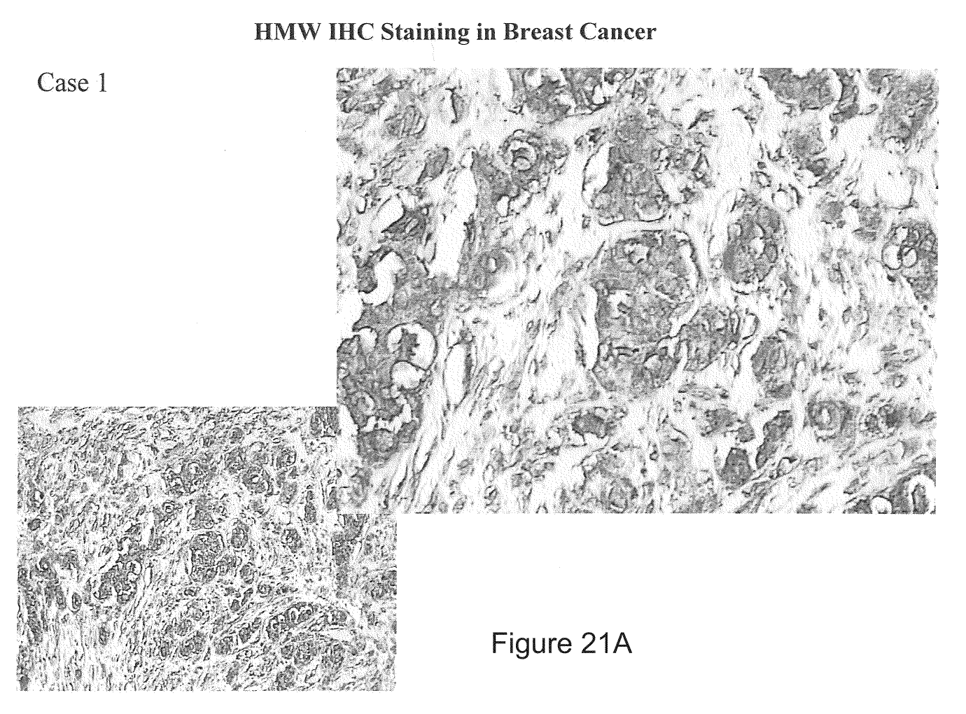

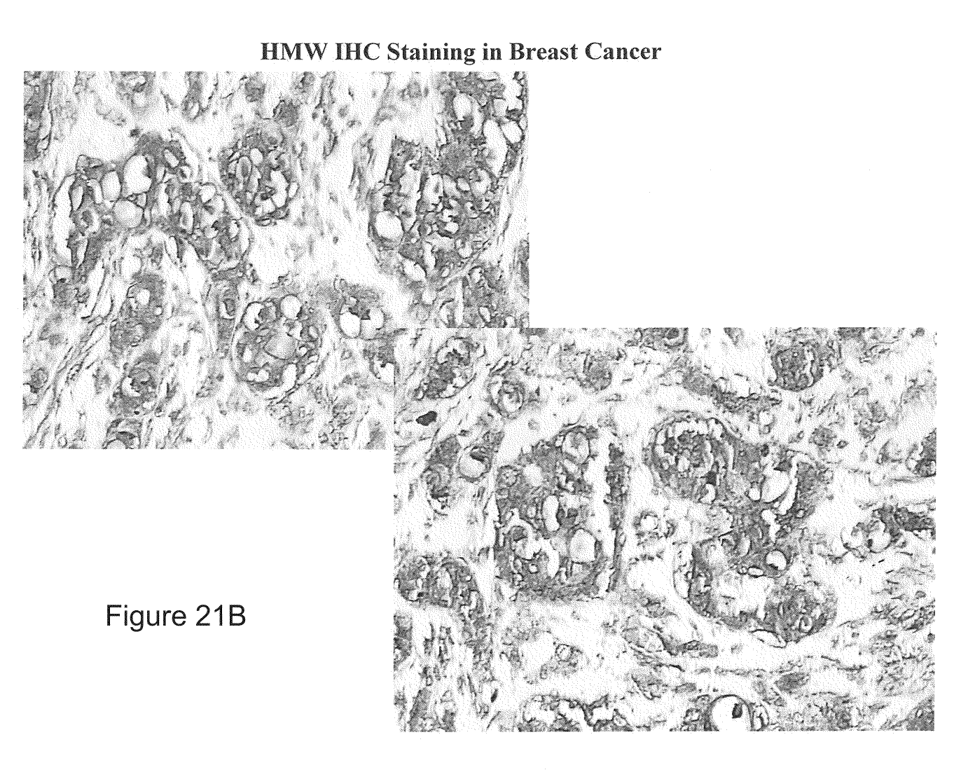

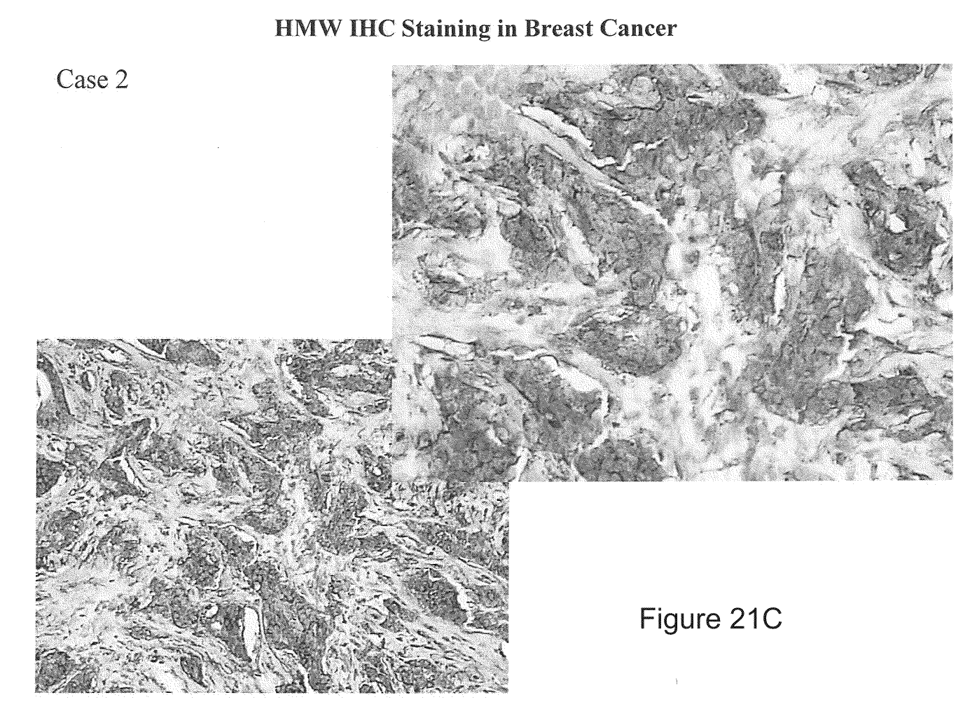

To further investigate the potential of HMW-MAA as a biomarker and validate the IHC, HMW-MAA mRNA was assessed by qRT. An optimal qRT assay for HMW-MAA detection was established using melanoma cell lines. HMW-MAA mRNA expression was measured by a qRT assay in 13 melanoma cell lines and compared to normal PBL (FIG. 2A). HMW-MAA mRNA expression was detectable in all 13 melanoma cell lines, but not in normal PBL. HMW-MAA mRNA detection was also performed in PEAT metastatic melanomas and normal LNs, and the assay conditions were optimized for qRT. HMW-MAA, MART-1, and GAPDH mRNA expression were measured by qRT in PEAT LN macrometastases, including SLN macrometastases, and SLN micrometastases. Previously, the MART-1 mRNA detection assay of SLN metastasis has been optimized in PEAT.⁸ Absolute mRNA copies of HMW-MAA, MART-1, and GAPDH ranged from 0 to 5.6×10⁵, from 0 to 7.0×10³, and from 1.1×10² to 7.5×10⁵, respectively. HMW-MAA mRNA was assessed in LN macrometastases and SLN micrometastases (FIG. 2B). Relative HMW-MAA mRNA copies were significantly higher in LN macrometastases (n=31, median=0.26) than in normal LN (P=0.0003). Relative HMW-MAA mRNA copies were also significantly higher in SLN micrometastases (n=17, median=0.06) than in normal LN (P=0.0033). In addition, relative HMW-MAA copies were higher in LN macrometastases than in SLN micrometastases (P=0.021). The cutoff was set for HMW-MAA positivity, and the HMW-MAA levels of 32 of 48 (67%) LN metastasis tissues were above cutoff. MART-1 mRNA was also assessed in the same specimens (FIG. 2C). Relative MART-1 copies were higher in LN macrometastases (P-0.0088) and SLN micrometastases (P=0.042) than in normal LN. MART-1 mRNA was detected in 31 of 48 (65%) LN metastases. HMW-MAA mRNA expression was detectable in LN metastases (12/48, 25%), whereas MART-1 mRNA expression was negative (data not shown). This finding is consistent with the IHC results. Both HMW-MAA and MART-1 mRNA were detected by qRT, and micrometastases were distinguishable from macrometastases by calculating the value of relative HMW-MAA copies.

The frequency of HMW-MAA and MART-1 mRNA expression was investigated by qRT in PEAT LN metastases (Table 4). HMW-MAA was expressed in 32 of 48 (67%) LN metastases and MART-1 was expressed in 31 of 48 (65%) LN metastases. The expression did not differ between HMW-MAA and MART-1 in LN metastases (NS). In addition, either HMW-MAA or MART-1 was expressed in 39 of 48 (81%) LN metastases. These results indicated that qRT sensitivity to HMW-MAA is equivalent to MART-1. Moreover, qRT of multiple markers (HMW-MAA and MART-1) may be more sensitive than that of a single marker.

TABLE 4
The Frequency of HMW-MAA and MART-1 mRNA Expression
Detected by qRT in LN Metastases

qRT

| | | |
|---|---|---|
| HMW-MAA | 48/48 (100) | 32/48 (67) |
| MART-1 | 41/48 (85) | 31/48 (65) |
| HMW-MAA or MART-1 | 48/48 (100) | 39/48 (81) |

TABLE 5
IHC of Primary and Distant Metastasis of Melanoma

| Stage of Disease | | HMW-MAA | | MART-1 | |
|---|---|---|---|---|---|
| Stage I Primary | | + | 75%< | +++ | 100% |
| Stage I Primary | | ++ | 100% | +++ | 100% |
| Stage II Primary | | ++ | 100% | ++ | 100% |
| Stage II Primary | | ++ | 100% | − | |
| Stage II Primary | | + | 75%< | ++ | 100% |
| Stage II Primary | | +++ | 100% | ++ | 100% |
| Stage II Primary | | ++ | 100% | ++ | 100% |
| Stage II Primary | | ++ | 50-75% | + | <25% |
| Stage II Primary | | + | <25% | +++ | 100% |
| Stage III Primary | | ++ | <25% | ++ | 25-50% |
| Stage III Primary | | ++ | 25-50% | + | 25-50% |
| Stage III Primary | | ++ | 75%< | + | 100% |
| Stage III Primary | | ++ | 75%< | +++ | 100% |
| Stage III Primary | | +++ | 75%< | +++ | 100% |
| Stage III Primary | | ++ | 75%< | +++ | 100% |
| Stage III Primary | | ++ | 75%< | − | |
| Stage IV Metastasis | small bowel | +++ | 100% | ++ | 50-75% |
| Stage IV Metastasis | skin | +++ | 100% | ++ | 75%< |
| Stage IV Metastasis | lung | − | | ++ | 50-75% |
| Stage IV Metastasis | small intestine | + | 75%< | + | 75%< |
| Stage IV Metastasis | lung | + | 75%< | + | <25% |
| Stage IV Metastasis | small bowel | ++ | 75%< | + | 100% |
| Stage IV Metastasis | skin | ++ | 100% | + | 100% |
| Stage IV Metastasis | left breast | +++ | 100% | + | 50-75% |
| Stage IV Metastasis | lymph node | +++ | 75%< | + | <25% |
| Stage IV Metastasis | thigh muscle | ++ | 75%< | + | 100% |
| Stage IV Metastasis | skin | ++ | 25-50% | − | |
| Stage IV Metastasis | skin | + | 75%< | − | |
| Stage IV Metastasis | lymph node | ++ | 75%< | ++ | 75%< |
| Stage IV Metastasis | lung | + | <25% | ++ | 100% |
| Stage IV Metastasis | lung | + | <25% | + | 100% |
| Stage IV Metastasis | colon | +++ | 100% | + | <25% |
| Stage IV Metastasis | lung | + | <25% | ++ | 100% |
| Stage IV Metastasis | lymph node | + | <25% | − | |
| Stage IV Metastasis | lymph node | + | <25% | − | |
| Stage IV Metastasis | skin | ++ | 75%< | + | 75%< |
| Stage IV Metastasis | lung | − | | + | 50-75% |
| Stage IV Metastasis | lymph node | ++ | 100% | + | 50-75% |
| Stage IV Metastasis | skin | ++ | 75%< | − | |
| Stage IV Metastasis | lymph node | +++ | 100% | + | 25-50% |
| Stage IV Metastasis | subcutaneous adipose | ++ | 50-75% | − | |
| Stage IV Metastasis | adductor magnus | + | <25% | − | |
| Stage IV Metastasis | lymph node | ++ | 100% | + | <25% |
| Stage IV Metastasis | skin | ++ | 75%< | − | |
| Stage IV Metastasis | pectoralis minor | +++ | 75%< | + | 25-50% |
| Stage IV Metastasis | jejunum | ++ | 100% | + | <25% |

### Discussion

More sensitive and accurate IHC biomarkers of detecting occult metastatic melanoma in SLNs may help reduce misdiagnosis of patients with risk of recurrence. In the present study, HMW-MAA mAb has been used for IHC of SLNs in melanoma patients. HMW-MAA mAb detected melanoma cells in all 84 LN metastases. IHC using HMW-MAA mAb was more sensitive and stained more intensely than IHC using MART-1 mAb, commonly used in current clinicopathology. Furthermore, HMW-MAA mAb detected occult tumor cells that were not detected by MART-1-specific Abs.

The S-100 protein is a small protein originally extracted from bovine brain and belongs to the family of calcium-binding proteins.²¹ S100 is a traditional IHC immunomarker for nevus and melanoma, expressed both in the cytoplasm and nucleus. However, S-100 lacks specificity, because S-100 is expressed in Langerhans cells, dendritic cells, macrophages, Schwann cells, and a wide range of tumors, such as peripheral nerve sheath and cartilaginous tumors, chordomas, histiocytosis X, Schwannomas, ependymomas, and astrogliomas.^{22,23} Several studies have used the anti-S-100 antibody for IHC diagnosis of primary and metastatic melanomas. In primary melanomas, the mean positive rate of IHC using S100-specific Ab was approximately 95% (range: 86-100).²⁴-27 Approximately 94% (range: 83-100) of metastatic melanomas expressed S-100.²⁴⁻²⁷

MB-45-specific mAb,²⁸ recognizes gp100 protein.^{29,30} Gp100 is a melanosomal matrix protein and melanoma antigen recognized by cytotoxic T lymphocytes and expressed in cytoplasm. HMB-45-specific mAb is also used in IHC for nevus and melanoma, but also stains breast carcinomas, plasmacytomas, angiomyolipomas, and pigmented nerve sheath tumors.³⁰ In primary melanomas, the mean positive rate of IHC using HMB-45-specific mAb was approximately 86% (range: 70-100).^{24,25,28,31} In metastatic melanomas, the mean IHC positive rate using HMB-45-specific mAb was approximately 72% (range: 43-100).^{24,25,31}

MART-1, also called Melan-A, is a small protein recognized as a target antigen by cytotoxic T lymphocytes.³² The Melan-A-specific mAb has been shown to stain the cytoplasm of both benign nevus cells and melanoma cells.^{31,33} Melan-A also stained positive in adrenocortical adenomas and carcinomas, and sex-cord stromal tumors of the ovary.³⁴ Many studies have shown MART-1 expression in primary and metastatic melanomas for IHC diagnosis. In primary melanoma lesions, the mean positive rate of MART-1 expression was approximately 84% (range: 75-97).^{24,27,31,33} Approximately 76% (range: 71-81) of metastatic melanomas expressed MART-1.^{24,27,31}

Among the three most common Abs for IHC, S-100, HMB-45, and MART-1 Abs, S-100 Ab has the highest sensitivity, but also the lowest specificity for melanoma.^{4,5,23} The sensitivity of both HMB-45 and MART-1 Abs is lower than S-100-specific Ab; and the expression of both HMB-45 and MART-1 are limited in tissues other than melanoma and nevus. Some studies have reported that MART-1 mAb is equivalent or more accurate to S-100 and HMB-45 Abs for evaluating melanoma micrometastases in SLNs.^{11,12} However, even MART-1 mAb cannot completely detect all melanomas.

IHC using HMW-MAA mAb was also performed for PEAT primary melanomas. In addition to primary melanoma cells, hair follicle cells, basal cells of the epidermis, and eccrine gland cells were detected by HMW-MAA-specific mAb (data not shown). However, HMW-MAA was not expressed in lymphocytes surrounding melanoma cells in LN metastases. Moreover, HMW-MAA proteins were expressed in all 84 LN metastasis tissues of melanoma, including SLN micrometastases.

Several RT-PCR or qRT studies using MART-1 specific primers and probe have previously been reported.^{8,19,20,35} In this study, HMW-MAA (67%) and MART-1 (65%) mRNA expression was detected via qRT in PEAT LN metastases. mRNA detection level in PEATs is lower than IHC detection level of the respective protein. Factors influencing mRNA detection could be the number of sections assessed, mRNA degradation, mRNA copy number, and fixation procedure of LNs. It is believed that HMW-MAA was as sensitive of an mRNA biomarker as MART-1 mRNA using qRT of PEATs for melanoma detection. Besides, HMW-MAA mRNA expression was detectable in LN metastases, whereas MART-1 mRNA expression was negative. By adding HMW-MAA into the qRT assay using MART-1 previously reported, the qRT assay sensitivity may increase. In addition, HMW-MAA relative copies, unlike MART-1, can distinguish SLN micrometastases from LN macrometastases. HMW-MAA is potentially a better mRNA marker to detect SLN micrometastases in melanoma patients.

In summary, the HMW-MAA-specific mAb cocktail used represents a useful biomarker to detect melanoma micrometastasis by IHC staining of SLN. Moreover, HMW-MAA is also a potential mRNA marker for detecting melanoma metastasis in PEAT SLN. These findings suggest that HMW-MAA has utility as a more sensitive and specific biomarker than current common biomarkers, and the use of HMW-MAA can improve occult tumor cell detection via IHC and qRT in SLNs of melanoma.

### REFERENCES

¹Morton D L, Wen D R, Wong J H, et al. Technical details of intraoperative lymphatic mapping for early stage melanoma. Arch Surg 127:392-9, 1992
²Bostick P, Essner R, Glass E, et al. Comparison of blue dye and probe-assisted intraoperative lymphatic mapping in melanoma to identify sentinel nodes in 100 lymphatic basins. Arch Surg 134:43-9, 1999
³Morton D L, Thompson J F, Essner R, et al. Validation of the accuracy of intraoperative lymphatic mapping and sentinel lymphadenectomy for early-stage melanoma: a multicenter trial. Multicenter Selective Lymphadenectomy Trial Group. Ann Surg 230:453-63; discussion 463-5, 1999
⁴Yu L L, Flotte T J, Tanabe K R, et al. Detection of microscopic melanoma metastases in sentinel lymph nodes. Cancer 86:617-27, 1999
⁵Cochran A J, Wen D R, Herschman H R. Occult melanoma in lymph nodes detected by antiserum to S-100 protein. Int J Cancer 34:159-63, 1984
⁶Messina J L, Glass L F, Cruse C W, et al. Pathologic examination of the sentinel lymph node in malignant melanoma. Am J Surg Pathol 23:686-90, 1999
⁷Gibbs J F, Huang P P, Zhang P J, et al. Accuracy of pathologic techniques for the diagnosis of metastatic melanoma in sentinel lymph nodes. Ann Surg Oncol 6:699-704, 1999
⁸Takeuchi H, Morton D L, Kuo C, et al. Prognostic significance of molecular upstaging of paraffin-embedded sentinel lymph nodes in melanoma patients. J Clin Oncol 22:2671-80, 2004
⁹Morton D L, Thompson J F, Cochran A J, Mozzillo N, Elashoff R, Essner R, Nieweg O E, Roses D F, Hoekstra H J, Karakousis C P, Reintgen D S, Coventry B J, Glass E C, Wang H J. MSLT Group. Sentinel-node biopsy or nodal observation in melanoma. N Engl J Med 355(13):1307-17, 2006. Erratum in: N Engl J Med 355(18):1944, 2006
¹⁰Campoli M R, Chang C C, Kageshita T, et al. Human high molecular weight-melanoma-associated antigen (HMW-MAA): a melanoma cell surface chondroitin sulfate proteoglycan (MSCP) with biological and clinical significance. Crit Rev Immunol 24:267-96, 2004
¹¹Shidham V B, Qi D Y, Acker S, et al. Evaluation of micrometastases in sentinel lymph nodes of cutaneous melanoma: higher diagnostic accuracy with Melan-A and MART-1 compared with S-100 protein and HMB-45. Am J Surg Pathol 25:1039-46, 2001
¹²Karimipour D J, Lowe L, Su L, et al. Standard immunostains for melanoma in sentinel lymph node specimens: which ones are most useful? J Am Acad Dermatol 50:759-64, 2004
¹³Chu K U, Turner R R, Hansen N M, et al. Do all patients with sentinel node metastasis from breast carcinoma need complete axillary node dissection? Ann Surg 229:536-41, 1999
¹⁴Carlson G W, Murray D R, Lyles R H, et al. The amount of metastatic melanoma in a sentinel lymph node: does it have prognostic significance? Ann Surg Oncol 10:575-81, 2003
¹⁵Sarantou T, Chi D D, Garrison D A, et al. Melanoma-associated antigens as messenger RNA detection markers for melanoma. Cancer Res 57:1371-6, 1997
¹⁶Takeuchi H, Fujimoto A, Tanaka M, et al. CCL21 chemokine regulates chemokine receptor CCR7 bearing malignant melanoma cells. Clin Cancer Res 10:2351-8, 2004
¹⁷Kim J, Reber H A, Hines 0 J, et al. The clinical significance of MAGEA3 expression in pancreatic cancer. Int J Cancer 118:2269-75, 2006
¹⁸Goto Y, Matsuzaki Y, Kurihara S, et al. A new melanoma antigen fatty acid-binding protein 7, involved in proliferation and invasion, is a potential target for immunotherapy and molecular target therapy. Cancer Res 66:4443-9, 2006
¹⁹Kuo C T, Hoon D S, Takeuchi H, et al. Prediction of disease outcome in melanoma patients by molecular analysis of paraffin-embedded sentinel lymph nodes. J Clin Oncol 21:3566-72, 2003
²⁰Takeuchi H, Kuo C, Morton D L, et al. Expression of differentiation melanoma-associated antigen genes is associated with favorable disease outcome in advanced-stage melanomas. Cancer Res 63:441-8, 2003
²¹Moore B W. A soluble protein characteristic of the nervous system. Biochem Biophys Res Commun 19:739-44, 1965
²²Nakajima T, Watanabe S, Sato Y, et al. Immunohistochemical demonstration of S100 protein in malignant melanoma and pigmented nevus, and its diagnostic application. Cancer 50:912-8, 1982
²³Cochran A J. Melanoma metastases through the lymphatic system. Surg Clin North Am 80(6):1683-93, 2000
²⁴Blessing K, Sanders D S, Grant J J. Comparison of immunohistochemical staining of the novel antibody melan-A with S100 protein and HMB-45 in malignant melanoma and melanoma variants. Histopathology 32:139-46, 1998
²⁵Fitzgibbons P L, Chaurushiya P S, Nichols P W, et al. Primary mucosal malignant melanoma: an immunohistochemical study of 12 cases with comparison to cutaneous and metastatic melanomas. Hum Pathol 20:269-72, 1989
²⁶Fujita S, Takahashi H, Tsuda N, et al. Immunohistochemical localization of S-100 protein and its subunits in melanotic lesions in the oral mucosa and skin. J Oral Pathol Med 20:429-32, 1991
²⁷Yu C H, Chen H H, Liu C M, et al. HMB-45 may be a more sensitive maker than S-100 or Melan-A for immunohistochemical diagnosis of primary oral and nasal mucosal melanomas. J Oral Pathol Med 34:540-5, 2005
²⁸Gown A M, Vogel A M, Hoak D, et al. Monoclonal antibodies specific for melanocytic tumors distinguish subpopulations of melanocytes. Am J Pathol 123:195-203, 1986
²⁹Hirano T. Immunohistochemical study of malignant melanoma. Acta Pathol Jpn 36:733-43, 1986
³⁰Prasad M L, Jungbluth A A, Iversen K, et al. Expression of melanocytic differentiation markers in malignant melanomas of the oral and sinonasal mucosa. Am J Surg Pathol 25:782-7, 2001
³¹Jungbluth A A, Busam K J, Gerald W L, et al. A103: An anti-melan-a monoclonal antibody for the detection of malignant melanoma in paraffin-embedded tissues. Am J Surg Pathol 22:595-602, 1998
³²Kawakami Y, Eliyahu S, Delgado C H, et al. Cloning of the gene coding for a shared human melanoma antigen recognized by autologous T cells infiltrating into tumor. Proc Natl Acad Sci USA 91:3515-9, 1994
³³Busam K J, Chen Y T, Old L J, et al. Expression of melan-A (MART1) in benign melanocytic nevi and primary cutaneous malignant melanoma. Am J Surg Pathol 22:976-82, 1998
³⁴Busam K J, Iversen K, Coplan K A, et al. Immunoreactivity for A103, an antibody to melan-A (Mart-1), in adrenocortical and other steroid tumors. Am J Surg Pathol 22:57-63, 1998
³⁵Koyanagi K, O'Day S J, Gonzalez R, et al. Serial monitoring of circulating melanoma cells during neoadjuvant biochemotherapy for stage III melanoma: outcome prediction in a multicenter trial. J Clin Oncol 23:8057-64, 2005

### Example III

### Beads Technique Procedure for Using HMW-MAA Antibodies

### Objective:

To obtain circulating tumor cells which express HMW-MAA using Beads.

### Materials:

Eight HMW-MAA Antibodies kept in refrigerator
mAbs 225.28, 763.74, VT80.12, VF4-TP108, VF1-TP41.2, VF20-VT5.1, and TP61.5

CELLection™ Pan Mouse IgG Kit (Prod. No.: 115.31)
Contains: 5 ml CELLection™ Pan Mouse IgG Dynabeads
   Releasing Buffer Component 1
   Releasing Buffer Component 2

DYNAL BIOTECH rotator

DYNAL MPC-S magnet

PBS and PBS with 0.1% BSA

1.5 ml eppendorf tube and 15 ml screw cap conical tube

Dry-bath incubator (heat block)

### Procedures:

1. Blood samples are provided as pellet in 15 ml screw cap conical tubes.

2. Suspend the PBL pellet with 4 ml PBS with 0.1% BSA (PBS/0.1% BSA) with same tube.

3. Add antibodies from SB04-423, SB04-424, SB04-425, SB04-426, SB04-427, SB05-674, SB05-675, and SB05-676, 3 ul each.

4. Incubate with 22 rpm rotation at 4° C. over night in a cold room.

5. Dynabeads washing procedure (CELLection™ Pan Mouse IgG Kit) on the next day:
a. Resuspend the Dynabeads in the vial by vortex.
b. Transfer the desired volume of Dynabeads to a new 1.5 ml eppendorf tube. Use 25 ul beads per sample unit (25 ul beads for <2.5×10⁶ cells).
c. Add 1 ml PBS and mix by gently pipetting.
d. Place the tube in a magnet for 1 min and discard the supernatant by 1000 ul pipette.
e. Remove the tube from the magnet and suspend the Dynabeads by the same volume of PBS as 5-b.

6. Take the sample from cold room and centrifuge at 1000 rpm at 4° C. for 10 min.

7. Discard the supernatant by 1000 ul pipette.

8. Suspend the pellet with 1.5 ml PBS/0.1% BSA and transfer to a new 1.5 ml eppendorf tube.

9. Add 25 ul Dynabeads and incubate with 22 rpm rotation at 4□ for 40 min(cold room)

10. Take the sample from the cold room and place the tube in a magnet for 1 min.

11. Transfer the supernatant by 1000 ul pipette to a new 15 ml screw cap conical tube. And keep it as “PBL”.

12. Remove the tube from the magnet.

13. Add 1.5 ml 37° C. PBS/0.1% BSA and place the tube in a magnet for 1 min.

14. Transfer the supernatant to the same 15 ml tubes as “PBL”. At last, this PBL tube may contain 3025 ul. This tube goes to step 29.

15. Remove the tube from the magnet.

16. Suspend the bead fraction with 300 μl 37° C. PBS/0.1% BSA.

17. Add 4 ul Beads Releasing Buffer per sample unit (kept in the freezer).

18. Incubate with 22 rpm rotation at room temperature for 20 min.

19. Pipette vigorously by 200 ul pipette 10 times.

20. Place in a magnet for 2 min.

21. Transfer the supernatant into a new 1.5 ml eppendorf tube.

22. Suspend the bead fraction with 300 ul 37° C. PBS/0.1% BSA.

23. Pipette vigorously by 200 ul pipette 10 times.

24. Place in a magnet for 2 min.

25. Transfer the supernatant into the same 1.5 ml eppendorf tube. At last, this tube may contain 600 ul.

26. Centrifuge at 2000 rpm for 5 min.

27. Discard the supernatant by pipette. Pellet should be captured tumor cells.

28. Suspend with 1 ml Tri-Reagent for RNA extraction. Go to step 33.

29. Centrifuge “PBL” 15 ml conical tubes at 1000-1500 rpm for 10 min.

30. Discard the supernatant by pipette. Pellet should be PBL.

31. Suspend with 1 ml Tri-Reagent and transfer to 1.5 ml tube for RNA extraction.

32. One “captured cell” tube and one “PBL” tube suspended with Tri-Reagent are obtained.

33. Go to RNA extraction.

The contents of all references cited herein are incorporated by reference in their entirety.

What is claimed is:

1. A method of isolating cells expressing the HMW-MAA protein, comprising: providing a device of claim 7 and a sample containing cells that express the HMW-MAA protein; contacting the device with the sample to allow binding of the HMW-MAA protein to its antibodies; and isolating the cells that express the HMW-MAA protein from the sample.

2. The method of claim 1, wherein the sample is a cancer tissue sample or a sample of a body fluid containing cancer cells.

3. The method of claim 1, further comprising analyzing a DNA, mRNA, or protein marker in the isolated cells.
